(19) United States
(12) Patent Application Publication
(10) Pub. No.: US 2004/0102524 A1
(43) Pub. Date: May 27, 2004

(54) Method of treatment

(76) Inventor: Andrew Hughes, Macclesfield (GB)

Correspondence Address: ASTRA ZENECA PHARMACEUTICALS LP, GLOBAL INTELLECTUAL PROPERTY, 1800 CONCORD PIKE, WILMINGTON, DE, 19850-5437, US

(21) Appl. No.: 10/469,358

(22) PCT Filed: Mar. 13, 2002
(86) PCT No.: PCT/SE02/00477

(30) Foreign Application Priority Data
Mar. 13, 2001 (SE) 0100873-9

(51) Int. Cl. A61K 31/137

(52) U.S. Cl. 514/651

(57) ABSTRACT

This invention relates to therapeutic agents, and in particular to the use of compounds such as EP1 antagonists, for reducing uric acid levels in a warm-blooded animal, such as a human. Provided herein is a method of reducing uric acid in a warm-blooded animal comprising administering to said animal an effective amount of an EP1 antagonists. EP1 antagonists are compounds which are antagonists of E-type prostaglandins.

This invention relates to therapeutic agents, and in particular to a method of using compounds such as EP1 antagonists, for reducing uric acid levels in a warm-blooded animal, such as a human.

Uric acid is a naturally occurring organic compound in a warm-blooded animal, occurring as a product of the metabolism of naturally occurring purines. Hyperuricemia, or raised uric acid concentrations in serum or plasma, may result from diminished renal excretion of uric acid, from elevated biosynthesis of uric acid or from abnormal metabolic (either anabolic or catabolic) processes. The disorder can be congenital, occurring secondary to inborn errors of metabolism, or may be acquired, as in elevated uric acid levels due to dietary composition, diminished excretion due to renal insufficiency, in neoplastic diseases such as leukemia, Burkitt's lymphoma and other cancers, or due to the cytotoxic effects of cancer chemotherapy or radiation therapy which result in an elevated level of products of cell lysis including purines which are metabolically converted to uric acid.

Inborn errors of metabolism include the Lesch-Nyhan syndrome, phosphoribosyl pyrophosphate synthetase overactivity and glucose-6-phosphatase deficiency.

A diminished renal excretion of uric acid can occur as a result of renal disease, certain drug therapies including administration of diuretics, and may accompany diseases such as hyperparathyroidism.

Increased production of uric acid is a known accompanying feature of administration of cytotoxic agents or of therapeutic radiation treatment as in the treatment of cancers, including lymphomas, leukemia, and solid tumors. This manifestation of hyperuricemia is a subset of a group of metabolic disorders collectively termed acute tumor lysis syndrome. As this relates to elevated uric acid levels, rapid lysis of cells results in an acute release into the circulation, of intracellular contents, including endogenous purines which are metabolic precursers to uric acid. These purines are converted by enzymes such as xanthine oxidase into uric acid.

The consequences of hyperuricemia include crystal arthropathy, gout and accompanying depositions of crystal deposits in other organs including tophi in the skin and tendons; and may occur as an associative factor in obesity, diabetes mellitus, hypertension, ischemic heart disease and hyperlipoproteinemia. Precipitation of uric acid, generally as crystals of monosodium urate in soft tissues is strongly associated with elevated levels of uric acid. The saturation level of uric acid in extracellular fluid at 37° C. has been measured at 0.4 mmol/L and thus is a target urate level for treatment of diseases associated with hyperuricemia such as gout wherein symptoms of the disease are mediated via crystalline deposits of uric acid or monosodium urate. Below this target level, preferably at levels from 0.25-0.35 mmol/L, crystalline deposits can effectively be redissolved and further crystallization can be prevented.

Diseases associated with elevated uric acid levels include, but are not limited to, gout, gouty arthritis, trophi, hyperuricemia associated with a congenital abnormality in the metabolism of xanthine oxidase, urate nephropathy incident to hyperuricemia, or hyperuricemia incident to therapeutic use of cytotoxic chemotherapy or radiation therapy.

The current treatment of hyperuricemia relies upon agents which either block the production or increase the elimination of uric acid. Allopurinol is commonly used to block the production of uric acid, although its use is limited by side effects, the most prominent being skin rashes and hypersensitivity which can be both disabling and at times life-threatening. As such, prescribers are warned to discontinue medication if a skin rash occurs. Hepatotoxicity can also accompany allopurinol use. Drugs which are used to increase the elimination of uric acid such as probenecid and sulfinopyrazone also suffer from similar side effects.

EP1 antagonists are compounds which are antagonists of E-type prostaglandins, in particular PGE₂. The EP1 receptor has been found to be involved in pain generation, and thus antagonists of the EP1 receptor have been sought as potential therapies for pain management.

The authors have surprisingly discovered that certain compounds, in particular, compounds known to be EP1 antagonists, have been demonstrated to rapidly and significantly reduce plasma concentrations of uric acid in humans.

Accordingly, the present invention provides a method of reducing uric acid levels in a warm-blooded animal comprising administering to said animal an effective amount of an EP1 antagonist.

The present invention also provides a method of treating hyperuricemia in a warm-blooded animal, comprising administering to said animal a therapeutically effective amount of an EP1 antagonist.

Further provided is a method of treating or preventing a disease caused by hyperuricemia in a warm-blooded animal, comprising administering to the animal suffering from said disease a therapeutically effective amount of an EP1 antagonist.

Typical EP1 antagonists useful in the practice of the current invention include the compounds described in WO 97/00863; WO 97/00864; WO 00/69465; EP0480641; EP 0534667; WO 96/03380; WO 96/06822; EPA 0733033; EPA 0847391; EPA 0835246 and EPA 0752421. The contents of the aforesaid European and International Patent Applications are hereby incorporated by reference.

Additional EP1 antagonists that can be used to practice the methods of the current invention are those described in U.S. Pat. No. 5,504,077; EP694546; U.S. Pat. No. 5,441,950; U.S. Pat. No. 5,420,270; U.S. Pat. No. 5,354,747; U.S. Pat. No. 5,354,746; U.S. Pat. No. 5,324,722; U.S. Pat. No. 5,304,644; U.S. Pat. No. 5,281,590; WO 9313082; EP539977; WO 9307132; EP512400; EP512399; EP218077; EP193822; U.S. Pat. No. 4,132,847; EP0878465; EP0300676; U.S. Pat. No. 4,775,680; EP0845451; EP0160408; U.S. Pat. No. 4,820,689 and WO 9827053. The contents of the aforesaid US, European and International Patents and Applications are hereby incorporated by reference.

Particular compounds that may be used in this invention include compounds of formula I and formula II:

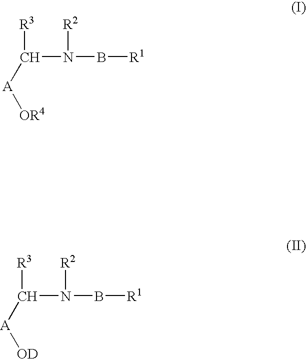

wherein:

A is an optionally substituted:

phenyl, naphthyl, pyridyl, pyrazinyl, pyridazinyl, pyrimidyl, thienyl, thiazolyl, oxazolyl or thiadiazolyl having at least two adjacent ring carbon atoms;

provided that the —CH(R³)N(R²)B—R¹ and —OR⁴ groups of formula I (or the OD group of formula II) are positioned in a 1,2 relationship to one another on ring carbon atoms and the ring atom positioned ortho to the OR⁴ linking group of formula I or the OD linking group of formula II (and therefore in the 3-position relative to the —CHR³NR²— linking group) is not substituted;

B is an optionally substituted:

phenyl, pyridyl, thiazolyl, oxazolyl, thienyl, thiadiazolyl, imidazolyl, pyrazinyl, pyridazinyl or pyrimidyl.

R¹ is positioned on ring B in a 1, 3 or 1,4 relationship with the —CH(R³)N(R²)— linking group and is carboxy, carboxyC₁₋₃alkyl, tetrazolyl, tetrazolylC₁₋₃alkyl, tetronic acid, hydroxamic acid, sulfonic acid, or R¹ is of the formula —CONR^{a} R^{a1} wherein R^{a} is hydrogen or C₁₋₆alkyl and R^{a1} is hydrogen, C₁₋₆alkyl (optionally substituted by halo, amino, C₁₋₄alkylamino, di-CIA alkylamino, hydroxy, nitro, cyano, trifluoromethyl, C₁₋₄alkoxy or C₁₋₄alkoxycarbonyl), C₂₋₆alkenyl (provided the double bond in not in the 1-position), C₂₋₆alkynyl (provided the triple bond is not in the 1-position), carboxyphenyl, 5- or 6-membered heterocyclylC₁₋₃alkyl, 5- or 6-membered heteroarylC₁₋₃alkyl, 5- or 6-membered heterocyclyl, or 5- or 6-membered heteroaryl or R^{a} and R^{a1} together with the amide nitrogen to which they are attached (NR^{a}R^{a1}) form an amino acid residue or ester thereof, or R¹ is of the formula —CONHSO₂R^{b} wherein R^{b} is C₁₋₆alkyl (optionally substituted by halo, hydroxy, nitro, cyano, trifluoromethyl, C₁₋₄alkoxy, amino, C₁₋₄alkylamino, di-C₁₋₄alkylamino or C₁₋₄alkoxycarbonyl), C₂₋₆alkenyl (provided the double bond is not in the 1-position), C₂₋₆alkynyl (provided the triple bond is not in the 1-position), 5- or 6-membered heterocyclylC₁₋₃alkyl, 5- or 6-membered heteroarylC₁₋₃alkyl, phenylC₁₋₃alkyl, 5- or 6-membered heterocyclyl, 5- or 6-membered heteroaryl or phenyl;

wherein any heterocyclyl or heteroaryl group in R^{a1} is optionally substituted by halo, hydroxy, nitro, cyano, trifluoromethyl, C₁₋₄alkoxy or C₁₋₄alkoxycarbonyl and any phenyl, heterocyclyl or heteroaryl group in R^{b} is optionally substituted by halo, trifluoromethyl, nitro, hydroxy, amino, cyano, C₁₋₆alkoxy, C₁₋₆alkylS(O)ₚ-(p is 0, 1 or 2), C₁₋₆alkyl carbamoyl, C₁₋₄alkylcarbamoyl, di(C₁₋₄alkyl)carbamoyl, C₂₋₆alkenyl, C₂₋₆alkynyl, C₁₋₄alkoxycarbonylamino, C₁₋₄alkanoylamino, C₁₋₄alkanoyl(N—C₁₋₄alkyl)amino, C₁₋₄alkanesulfonamido, benzenesulfonamido, aminosulfonyl, C₁₋₄alkylaminosulfonyl, di(C₁₋₄alkyl)aminosulfonyl, C₁₋₄alkoxycarbonyl, C₁₋₄alkanoyloxy, C₁₋₆alkanoyl, formylC₁₋₄alkyl, hydroxyiminoC₁₋₆alkyl, C₁₋₄alkoxyiminoC₁₋₆alkyl or C₁₋₆alkylcarbamoylamino; or R¹ is of the formula —SO2N(R^{c})R^{c1}, wherein R^{c} is hydrogen or C₁₋₄alkyl and R^{c1} is hydrogen or C₁₋₄alkyl;

or R¹ is of the formula (IA), (IB) or (IC):

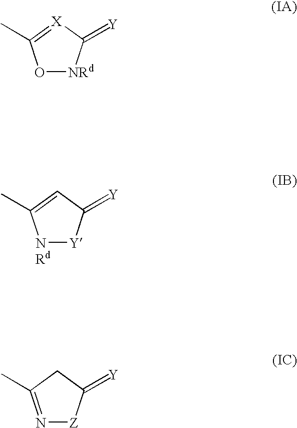

wherein X is CH or nitrogen, Y is oxygen or sulfur, Y′ is oxygen or NR^{d} and Z is CH₂, NR^{d} or oxygen provided that there is no more than one ring oxygen and there are at least two ring heteroatoms and wherein R^{d} is hydrogen or C₁₋₄alkyl.

R² is hydrogen, C₁₋₆alkyl, optionally substituted by hydroxy, cyano or trifluoromethyl, C₂₋₆alkenyl (provided the double bond is not in the 1-position), C₂₋₆alkynyl (provided the triple bond is not in the 1-position), phenylC₁₋₃alkyl or pyridylC₁₋₃alkyl.

R³ is hydrogen, methyl or ethyl.

R⁴ of formula I is optionally substituted: C₁₋₆alkyl, C₃₋₇cycloalkylC₁₋₃alkyl or C₃₋₇cycloalkyl;

and N-oxides of —NR where chemically possible;

and S-oxides of sulfur containing rings where chemically possible;

D of formula II is hydrogen, an optionally substituted 5-7 membered carbocyclic ring containing one double bond, C₁₋₃alkyl substituted by an optionally substituted 5-7 membered carbocyclic ring containing one double bond or D is of the formula—(CH₂)ₙCH(R⁵)C(R⁶)═C(R⁷)R⁸ wherein:

R⁵ is independently selected from hydrogen, methyl or ethyl;

R⁶ is hydrogen, methyl, bromo, chloro, fluoro or trifluoromethyl;

R⁷ is hydrogen, C₁₋₄alkyl, bromo, chloro, fluoro or trifluoromethyl;

R⁸ is hydrogen, C₁₋₄alkyl, bromo, chloro, fluoro or trifluoromethyl;

n is 0 or 1;

and N-oxides of —NR² where chemically possible;

and S-oxides of sulfur containing rings where chemically possible;

and pharmaceutically acceptable salts and in vivo hydrolysable esters and amides thereof;

A 5- or 6-membered heteroaryl ring system is a monocyclic aryl ring system having 5 or 6 ring atoms wherein 1, 2 or 3 ring atoms are selected from nitrogen, oxygen and sulfur.

A 5- or 6-membered heterocyclic ring is a ring system having 5 or 6 ring atoms wherein 1, 2 or 3 of the ring atoms are selected from nitrogen, oxygen and sulfur.

Particular 5- or 6-membered monocyclic heteroaryl rings include pyrrolyl, imidazolyl, pyrazolyl, isothiazolyl, isoxazolyl, pyridyl, pyrazinyl, pyrimidinyl, pyridazinyl, thiazolyl, thiadiazolyl, thienyl, furyl and oxazolyl.

Particular 5- or 6-membered heterocyclic ring systems include pyrrolidinyl, pyrrolinyl, imidazolidinyl, pyrazolidinyl, piperidyl, piperazinyl and morpholinyl.

Particular substituents for ring carbon atoms in A (heterocyclyl and heteroaryl rings include halo, trifluoromethyl, nitro, hydroxy, amino, C₁₋₄alkylamino, diC₁₋₄alkylamino, cyano, C₁₋₆alkoxy, C₁₋₆alkylS(O)ₚ-(p is 0, 1 or 2), C₁₋₆alkyl (optionally substituted by hydroxy, amino, halo, nitro or cyano), CF₃S(O)ₚ-(p=0, 1 or 2), carbamoyl, C₁₋₄alkylcarbamoyl, di(C₁₋₄alkyl)carbamoyl, C₂₋₆alkenyl, C₂₋₆alkynyl, C₁₋₄alkoxycarbonylamino, C₁₋₄alkanoylamino, C₁₋₄alkanoyl(N—C₁₋₄alkyl)amino, C₁₋₄alkanesulfonamido, benzenesulfonamido, aminosulfonyl, C₁₋₄alkylaminosulfonyl, C₁₋₄alkanoylaminosulfonyl, di(C₁₋₄alkyl)aminosulfonyl, C₁₋₄alkoxycarbonyl, C₁₋₄alkanoyloxy, C₁₋₆alkanoyl, formylC₁₋₄alkyl, trifluoroC₁₋₃alkylsulfonyl, hydroxyiminoC₁₋₆alkyl, C₁₋₄alkoxyiminoC₁₋₆alkyl and C₁₋₆alkylcarbamoylamino.

Where a ring nitrogen atom in A can be substituted without becoming quaternized, it is unsubstituted or substituted by C₁₋₄alkyl.

Particular substituents for ring carbon atoms in B include halo, trifluoromethyl, nitro, hydroxy, C₁₋₆alkoxy, C₁₋₆alkyl, amino, C₁₋₄alkylamino, di(C₁₋₄alkyl)amino, cyano, C₁₋₆alkyl S(O)ₚ-(p is 0, 1 or 2), carbamoyl, C₁₋₄alkylcarbamoyl and di(C₁₋₄alkyl)carbamoyl.

Where a ring nitrogen atom in B can be substituted without becoming quaternized, it is unsubstituted or substituted by C₁₋₄alkyl.

The term alkyl when used herein includes straight chain and branched chain substituents for example methyl, ethyl, n-propyl, isopropyl, n-butyl and isobutyl and functional groups on alkyl chains may be anywhere on the chain, for example hydroxyiminoC₁₋₆alkyl includes 1-(hydroxyimino)propyl and 2-(hydroxyimino)propyl.

C₁₋₆alkyl substituted by halo includes trifluoromethyl.

For the D moiety of compounds of formula II, particular substituents for the 5-7 membered carbocyclic ring containing one double bond include C₁₋₄alkyl, C₂₋₄alkenyl, C₂₋₄alkynyl, halo, hydroxy, amino, C₁₋₄alkylamino, di-(C₁₋₄alkyl)amino, cyano, trifluoromethyl, oxo, C₁₋₄alkanoyl, carboxy and carbamoyl.

Amino acid residues formed from R¹ and R^{a1} together with the nitrogen to which they are attached include residues (—NHCH(R)COOH) derived from naturally-occurring and non-naturally-occurring amino acids. Examples of, suitable amino acids include glycine, alanine, serine, threonine, phenylalanine, glutamic acid, tyrosine, lysine and dimethylglycine.

Suitable ring systems of the formula (IA), (IB), or (IC) include 5-oxo-4,5-dihydro-1,2,4-oxadiazol-3-yl, 3-oxo-2,3-dihydro-1,2,4-oxadiazol-5-yl, 3-thioxo-2,3-dihydro-1,2,4-oxadiazol-5-yl, 5-oxo-4,5-dihydro-1,3,4-oxadiazol-2-yl, 5-oxo-4,5-dihydro-1,2,4-triazol-3-yl, 5-thioxo-4,5-dihydro-1,3,4-oxadiazol-2-yl, 1,3,4-oxadiazol-2-yl, 3-hydroxy-2-methylpyrazol-5-yl, 3-oxo-2,3-dihydroisoxazol-5-yl, 5-oxo-1,5-dihydroisoxazol-3-yl and 5-oxo-2,3-dihydropyrazol-3-yl.

Examples of C₁₋₆alkoxycarbonyl are methoxycarbonyl, ethoxycarbonyl and t-butoxycarbonyl; examples of carboxyC₁₋₃alkyl are carboxymethyl, 2-carboxyethyl, 1-carboxyethyl and 3-carboxypropyl; examples of C₁₋₆alkoxycarbonylC₁₋₃alkyl are methoxycarbonylmethyl, ethoxycarbonylmethyl and methoxycarbonylethyl; examples of tetrazolylC₁₋₃alkyl are tetrazolylmethyl, and 2-tetrazolylethyl; examples of C₁₋₄alkoxy are methoxy, ethoxy, propoxy and isopropoxy; examples of C₂₋₆alkenyl are vinyl and allyl; examples of C₂₋₆alkynyl are ethynyl and propynyl; examples of C₁₋₄alkanoyl are formyl, acetyl, propionyl and butyryl; examples of halo are fluoro, chloro, bromo and iodo; examples of C₁₋₄alkylamino are methylamino, ethylamino, propylamino and isopropylamino; examples of di(C₁₋₄alkyl)amino are dimethyl amino, diethylamino and ethylmethylamino; examples of C₁₋₆alkylS(O)ₚ— are methylthio, methylsulfinyl and methylsulfonyl; examples of C₁₋₄alkylcarbamoyl are methylcarbamoyl and ethylcarbamoyl; examples of di(C₁₋₄alkyl)carbamoyl are dimethylcarbamoyl, diethylcarbamoyl and ethylmethylcarbamoyl; examples of C₁₋₆alkyl are methyl, ethyl, propyl and isopropyl; examples of C₁₋₄alkoxycarbonylamino are methoxycarbonylamino and ethoxycarbonylamino; examples of C₁₋₄alkanoylamino are acetamido and propionamido; examples of C₁₋₄alkanoyl(N—C₁₋₄alkyl)amino are N-methylacetamido and N-methylpropionamido; examples of C₁₋₄alkanesulfonamido are methanesulfonamido and ethanesulfonamido; examples of C₁₋₄alkylaminosulfonyl are methylaminosulfonyl and ethylaminosulfonyl; examples of di(C₁₋₄alkyl)aminosulfonyl are dimethylaminosulfonyl, diethylaminosulfonyl and ethylmethylaminosulfonyl; examples of C₁₋₄alkanoyloxy are acetyloxy and propionyloxy; examples of formylC₁₋₄alkyl are formylmethyl and 2-formylethyl; examples of hydroxyiminoC₁₋₆alkyl are hydroxyiminomethyl and 2-(hydroxyimino)ethyl; and examples of C₁₋₄alkoxyiminoC₁₋₆alkyl are methoxyiminomethyl, ethoxyiminomethyl and 2-(methoxyimino)ethyl.

It will also be understood that certain compounds of the present invention may exist in solvated, for example hydrated, as well as unsolvated forms. It is to be understood that the present invention encompasses all such solvated forms which possess the property of reducing uric acid levels.

Preferably A is optionally substituted: phenyl, naphthyl, thiadiazolyl, thienyl, pyridyl or pyrimidyl.

Preferably B is optionally substituted: pyridyl, phenyl, thiazolyl, thienyl, pyridazinyl, or oxazolyl.

Most preferably A is optionally substituted: phenyl or thienyl.

Most preferably B is optionally substituted: pyridyl, phenyl, thienyl, pyridazinyl or thiazolyl.

In particular A is optionally substituted phenyl.

In particular B is optionally substituted: pyrid-2,5-diyl, pyridazin-3,6-diyl, phen-1,4-diyl or thien-2,5-diyl.

Most particularly B is optionally substituted pyridazin-3,6-diyl or pyrid-2,5-diyl.

Most preferably B is pyridazinyl;

for compounds of formula II, when D is hydrogen, preferably B is optionally substituted: pyridyl, thienyl, pyridazinyl or thiazolyl;

Preferred optional substituents for ring carbon atoms in A, are halo, nitro, trifluoromethyl, cyano, amino, C₁₋₆alkoxy, carbamoyl, C₁₋₄alkylcarbamoyl, di(C₁₋₄alkyl)carbamoyl, C₁₋₄alkanoylamino, C₁₋₆alkylS(O)ₚ—, C₁₋₄alkanesulfonamido, benzenesulfonamido, C₁₋₆alkanoyl, C₁₋₄alkoxyiminoC₁₋₄alkyl and hydroxyiminoC₁₋₄alkyl.

Preferably, when A is a 6-membered ring, A is unsubstituted or substituted in the 4-position relative to the —OR⁴ group-of compounds of formula I or the OD group of compounds of formula II.

Preferred optional substituents for ring carbon atoms of B are halo, trifluoromethyl, C₁₋₄alkyl, amino, C₁₋₄ alkylamino, diC₁₋₄ alkylamino, nitro, hydroxy, C₁₋₆alkoxy and cyano.

For compounds of formula II, preferably n is 0;

Preferably A is unsubstituted or substituted by one substituent.

More preferably A is unsubstituted or substituted by bromo, methanesulfonyl, fluoro or chloro.

Most preferably A is unsubstituted or substituted by bromo or chloro.

Preferably B is unsubstituted or substituted by one substituent.

Most preferably B is unsubstituted.

Preferably R¹ is carboxy, carbamoyl or tetrazolyl or R¹ is of the formula —CONR^{a}R^{a1} wherein R^{a} is hydrogen or C₁₋₆alkyl and R^{a1} is C₁₋₆alkyl optionally substituted by hydroxy, C₂₋₆alkenyl, 1-morpholinyl, 1-piperidinyl, 1-pyrrolidinyl, pyridylC₁₋₃alkyl or R¹ is of the formula —CONHSO₂R^{b} wherein R^{b} is optionally substituted:

C₁₋₆alkyl, phenyl or 5- or 6-membered heteroaryl.

In particular, R¹ is carboxy, tetrazolyl or of the formula —CONR^{a} R^{a1} wherein R^{a} is hydrogen and R^{a1} is C₁₋₆alkyl optionally substituted by hydroxy or pyridylmethyl, or R¹ is of the formula —CONHSO₂R^{b} wherein R^{b} is C₁₋₆alkyl (optionally substituted by hydroxy or fluoro), phenyl (optionally substituted by acetamido), isoxazolyl (optionally substituted by methyl) or 1,3,4-thiadiazolyl (optionally substituted by acetamido).

Most preferably R¹ is carboxy, tetrazole or of the formula —CONHR^{a1} wherein R^{a1} is pyridylmethyl or C₁₋₄alkyl optionally substituted by hydroxy, or of the formula —CONHSO₂R^{b} wherein R^{b} is C₁₋₄ alkyl, 3,5-dimethylisoxazol-4-yl, or 5-acetarmido-1,3,4-thiadiazol-2-yl.

In another aspect R¹ is carboxy, carbamoyl or tetrazolyl or R¹ is of the formula —CONR^{a} R^{a1} wherein R^{a} is hydrogen or C₁₋₆alkyl and R^{a1} is C₁₋₆alkyl optionally substituted by hydroxy, C₂₋₆alkenyl, 1-morpholinyl, 1-piperidinyl, 1-pyrrolidinyl, pyridylC₁₋₃alkyl or R¹ is of the formula —CONHSO₂R^{b} wherein R^{b} is C₁₋₆alkyl or phenyl.

Preferably R² is hydrogen, methyl, ethyl, 2,2,2-trifluoroethyl, cyanomethyl, allyl or 3-propynyl.

More preferably R² is hydrogen, methyl, ethyl or propyl.

Yet more preferably R² is hydrogen or ethyl.

Most preferably R² is ethyl.

Preferably R³ is hydrogen.

Preferably R⁴ is optionally substituted by halo, hydroxy, C₁₋₄alkoxy, amino, carboxy, C₁₋₄ alkylS(O)ₚ-(p=0, 1 or 2), carbamoyl, trifluoromethyl, oxo or cyano.

More preferably R⁴ is optionally substituted by fluoro, chloro or bromo.

Most preferably R⁴ is optionally substituted by fluoro, trifluoromethyl, cyano or hydroxy.

Preferably R⁴ is C₁₋₄alkyl, C₃₋₆cycloalkyl or C₃₋₆cycloalkylmethyl.

More preferably R⁴ is propyl, isobutyl, butyl, 2-ethylbutyl, 2(R)-methylbutyl, 2(S)-methylbutyl, 2,2,2-trifluoroethyl, cyclopentylmethyl, cyclopropylmethyl, cyclopropyl or cyclopentyl.

Most preferably R⁴ is propyl, isobutyl, butyl, 2-ethylbutyl, cyclopentyl, cyclopropylmethyl or cyclopropyl;

for compounds of formula II,

Preferably R⁵ is hydrogen or methyl.

Preferably R⁶ is hydrogen, methyl or chloro.

Preferably R⁷ is hydrogen, methyl or chloro.

Preferably R⁸ is hydrogen or methyl.

Preferably the 5-7 membered carbocyclic ring containing one double bond is optionally substituted by methyl.

More preferably the 5-7 membered carbocyclic ring containing one double bond is unsubstituted.

Preferably D is a 5-6 membered carbocyclic ring containing one double bond (optionally substituted by methyl) methyl substituted by a 5-6 membered carbocyclic ring containing one double bond (optionally substituted by methyl) or of the formula —CH₂C(R⁶)═C(R⁷)R⁸.

Most preferably D is of the formula:

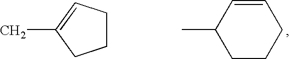

—CH₂CH═CH₂, —CH₂CH═CHMe, —CH₂CH═C(Me)₂, —CH₂C(Me)═CHMe, —CH₂C(Me)═CHMe, —CH₂C(Me)═CH₂ or —CH₂C(Cl)═CH₂.

In one aspect D is an optionally substituted 5-7 membered carbocyclic ring containing one double bond, C₁₋₃alkyl substituted by a 5-7 membered carbocyclic ring or of the formula —(CH₂)ₙCHR⁵C(R⁶)═C(R⁷)R⁸.

In another aspect D is hydrogen.

A preferred class of compounds is that of the formula IE or formula IIE:

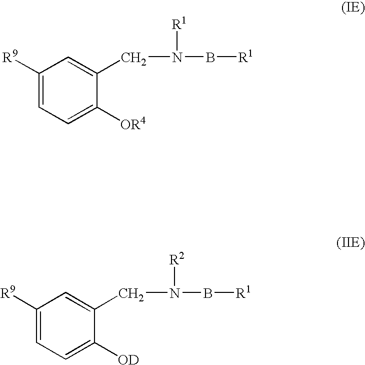

wherein

R¹ and R² and D are as hereinabove defined, R⁴ of formula I is C₁₋₄alkyl, C₃₋₆cycloalkyl or C₃₋₆ cycloalkylmethyl, R⁹ is hydrogen or as hereinabove defined for substituents for ring carbon atoms in A, and B is phenyl, thienyl, pyridazinyl, pyridyl, or thiazolyl.

Compounds of Formula I, for use in this invention are described in WO 97/00863 which is incorporated by reference herein.

Preferred compounds of formula I for use in this invention are:

N-propanesulfonyl-6-[N-(5-bromo-2-(cyclopropylmethoxy)benzyl)-N-ethylamino]pyridazine-3-carboxamide (described as Example 14 in International Patent Application WO 97/00863);

N-(3,5-dimethylisoxazol-4-ylsulfonyl)-6-[N-(5-chloro-2-(2-methylpropoxy)benzyl)-N-ethylamino]pyridazine-3-carboxamide (described as compound number I in Example 8 in International Patent Application WO 97/00863); and

6-[N-(5-bromo-2-(cyclopropylmethoxy)benzyl)-N-ethylamino]pyridazine-3-carboxylic acid (described as Example 3 in International Patent Application WO 97/00863).

Compounds of formula II for use in this invention are described in WO 97/00864 which is incorporated herein by reference.

A preferred compound of formula II for use in the present invention is:

6-[N-(5-bromo-2-(2-methylprop-2-en-1-yloxy)benzyl)-N-ethylamino]pyridazine-3-carboxylic acid (described as Example 15 in International Patent Application WO 97/00864).

Additional compounds which may be used in the practice of the present invention are compounds of the formula III;

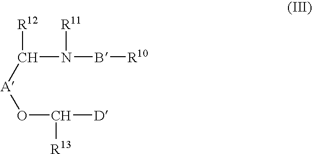

wherein:

A′ is an optionally substituted:

phenyl, naphthyl, pyridyl, pyrazinyl, pyridazinyl, pyrimidyl, thienyl, thiazolyl, oxazolyl, thiadiazolyl having at least two adjacent ring carbon atoms or a bicyclic ring system of the formula:

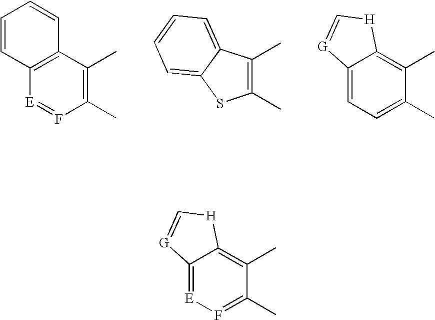

wherein E is nitrogen or CH, F is nitrogen or CH, G is sulphur or oxygen and H is nitrogen or CH;

provided that the —CH(R¹²)N(R¹¹)B—R¹⁰ and —OCH(R¹³)-D′ linking groups are positioned in a 1,2 relationship to one another on ring carbon atoms and the ring atom positioned ortho to the —OCHR¹³— linking group (and therefore in the 3-position relative to the —CHR¹²NR¹¹— linking group) is not substituted;

B′ is an optionally substituted:

phenyl, pyridyl, thiazolyl, oxazolyl, thienyl, thiadiazolyl, isoxazole, pyrazole, furyl, pyrrolyl, imidazolyl, pyrazinyl, pyridazinyl, pyrimidyl, pyridone, pyrimidone, pyrazinone or pyridazinone;

D′ is optionally substituted: pyridyl, pyrazinyl, pyrimidinyl, pyridazinyl, pyrrolyl, thienyl, furyl, imidazolyl, pyrazolyl, thiazolyl, isothiazolyl, oxazolyl, isoxazolyl or phenyl;

R¹⁰ is positioned on ring B′ in a 1, 3 or 1,4 relationship with the —CH(R¹²)N(R¹¹) linking group in 6-membered rings and in a 1,3-relationship with the —CH(R¹²)N(R¹¹)— linking group in 5-membered rings and is carboxy, carboxyC₁₋₃alkyl, tetrazolyl, tetrazolylC₁₋₃alkyl, tetronic acid, hydroxamic acid, sulphonic acid, or R¹⁰ is of the formula (IIIA), (IIIB) or (IIIC):

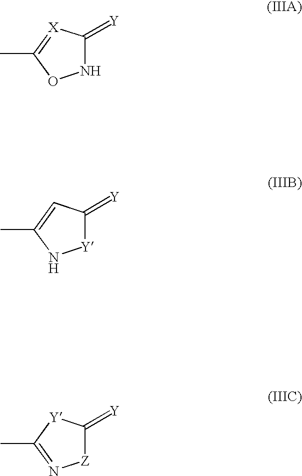

wherein X is CH or nitrogen, Y is oxygen or sulphur Y¹ is oxygen or NH, and Z is CH₂, NH or oxygen provided that there is no more than one ring oxygen and there are at least two ring heteroatoms; or R¹⁰ is of the formula —CONR^{e} R^{e1} or —C₁₋₃alkylCONR^{e}R^{e1} wherein R^{e} is hydrogen, C₁₋₆alkyl, C₃₋₇cycloalkyl, C₃₋₇cycloalkylC₁₃alkyl, C₅₋₇cycloalkenyl or C₅₋₇cycloalkenylC₁₋₃alkyl and R^{e1} is hydrogen, hydroxy or optionally substituted: C₁₋₁₀alkyl, C₁₋₁₀alkenyl, C₁₋₁₀alkynyl, C₃₋₇cycloalkyl, C₃₋₇cycloalkylC₁₋₆alkyl, C₃₋₇cycloalkylC₂₋₆alkenyl, C₃₋₇cycloalkylC₂₋₆alkynyl, C₅₋₇cycloalkenyl, C₃₋₇cycloalkenylC₁₋₆alkyl, C₅₋₇cycloalkenylC₂₋₆alkenyl, C₅₋₇cycloalkenylC₂₋₆alkynyl, 5- or 6-membered heteroaryl, 5- or 6-membered heteroarylC₁₋₆alkyl, 5- or 6-membered saturated or partially saturated heterocyclyl or 5- or 6-membered saturated or partially saturated heterocyclylC₁₋₆alkyl; or wherein R^{e} and R^{e1} together with the amide nitrogen to which they are attached (NR^{e}R^{e1}) form an amino acid residue or ester thereof; or R¹⁰ is of the formula —CONHSO₂R^{f} or —C₁₋₃alkylCONHSO₂R^{f} wherein R^{f} is optionally substituted: C₁₋₁₀alkyl, C₂₋₁₀alkenyl, C₂₋₁₀alkynyl, C₃₋₇cycloalkyl, C₃₋₇cycloalkylC₁₋₆alkyl, C₃₋₇cycloalkylC₂₋₆alkenyl, C₃₋₇cycloalkylC₂₋₆alkynyl, C₅₋₇cycloalkenyl, C₃₋₇cycloalkenylC₁₋₆alkyl, C₅₋₇cycloalkenylC₂₋₆alkenyl, C₅₋₇cycloalkenylC₂₋₆alkynyl, 5- or 6-membered heteroaryl, 5- or 6-membered heteroylarC₁₋₆alkyl, phenyl, phenylC₁₋₆alkyl, 5- or 6-membered saturated or partially saturated heterocyclyl or 5- or 6-membered saturated or partially saturated heterocyclylC₁₋₆alkyl or R¹⁰ is of the formula —CONR^{e}N(R^{g})R^{h} or —C₁₋₃alkylCONR^{e}N(R^{g})R^{h} wherein R^{e} is as hereinabove defined, R^{g} is hydrogen or C₁₋₆alkyl and R^{h} is hydrogen, hydroxy or optionally substituted: C₁₋₁₀alkyl, C₂₋₁₀alkenyl, C₂₋₁₀alkynyl, C₃₋₇cycloalkyl, C₃₋₇cycloalkylC₁₋₆alkyl, C₃₋₇cycloalkylC₂₋₆alkenyl, C₃₋₇cycloalkylC₂₋₆alkynyl, C₅₋₇cycloalkenyl, C₅₋₇cycloalkenylC₁₋₆alkyl, C₅₋₇cycloalkenylC₂₋₆alkenyl, C₅₋₇cycloalkenylC₂₋₆alkynyl, 5- or 6-membered heteroaryl, 5- or 6-membered heteroarylC₁₋₆alkyl, 5- or 6-membered saturated or partially saturated heterocyclyl, 5- or 6-membered saturated or partially saturated heterocyclylC₁₋₆alkyl, or R^{g} and R^{h}, together with the nitrogen atom to which they are attached, form a 4 to 8-membered saturated or partially saturated heterocyclic ring or form an amino acid residue or ester thereof;

R¹¹ is hydrogen, C₁₋₆alkyl (optionally substituted by hydroxy, cyano, nitro, amino, halo, C₁₋₄alkanoyl, C₁₋₄alkoxy or trifluoromethyl) C₂₋₆alkenyl, C₂₋₆alkynyl, C₃₋₆cycloalkyl, C₃₋₆cycloalkylC₁₋₃alkyl, C₃₋₆cycloalkylC₂₋₃alkenyl, C₅₋₆cycloalkenyl, C₅₋₆cycloalkenylC₁₋₃alkyl, C₅₋₆cycloalkenylC₂₋₃alkenyl, phenylC₁₋₃alkyl or 5- or 6-membered heteroarylC₁₋₃alkyl;

R¹² is hydrogen or C₁₋₄alkyl;

R¹³ is hydrogen or C₁₋₄alkyl;

and N-oxides of —NR¹¹ where chemically possible;

and S-oxides of sulphur containing rings where chemically possible;

and pharmaceutically acceptable salts and in vivo hydrolysable esters and amides thereof.

A 5- or 6-membered heteroaryl ring system is a monocyclic aryl ring system having 5 or 6 ring atoms wherein 1, 2 or 3 ring atoms are selected from nitrogen, oxygen and sulphur.

A 5- or 6-membered saturated or partially saturated heterocyclic (heterocyclyl) ring is a ring system having 5 or 6 ring atoms wherein 1, 2 or 3 of the ring atoms are selected from nitrogen, oxygen and sulphur.

Particular 5- or 6-membered monocyclic heteroaryl rings include pyrrolyl, imidazolyl, pyrazolyl, isothiazolyl, isoxazolyl, pyridyl, pyrazinyl, pyrimidinyl, pyridazinyl, thiazolyl, thiadiazolyl, thienyl, furyl and oxazolyl.

Particular 5- or 6-membered saturated or partially saturated heterocyclic ring systems include pyrrolidinyl, pyrrolinyl, imidazolidinyl, pyrazolidinyl, piperidyl, piperazinyl and morpholinyl.

Particular substituents for ring carbon atoms in A′ include halo, trifluoromethyl, nitro, hydroxy, amino, C₁₋₄alkylamino, diC₁₋₄alkylamino, cyano, C₁₋₆alkoxy, S(O)ₚC₁₋₆alkyl (p is 0, 1 or 2), C₁₋₆alkyl (optionally substituted by hydroxy, amino, halo, nitro or cyano), S(O)ₚCF₃ (p=0, 1 or 2), carbamoyl, C₁₋₄alkylcarbamoyl, di(C₁₋₄alkyl)carbamoyl, C₂₋₆alkenyl, C₂₋₆alkynyl, C₂₋₄alkenylamino, N—C₂₋₄alkenyl-N—C₁₋₄alkylamino, di-C₂₋₄alkenylamino, S(O)ₚC₂₋₆alkenyl, C₂₋₄alkenylcarbamoyl, N—C₂₋₄alkenyl-N-alkylamino, di-C₂₋₄alkenylcarbamoyl, C₃₋₇cycloalkyl, C₃₋₇cycloalkylC₁₋₃alkyl, C₃₋₇cycloalkylC₂₋₃alkenyl, C₅₋₇cycloalkenyl, C₅₋₇cycloalkenylC₁₋₃alkyl, C₅₋₇cycloalkenylC₂₋₃alkenyl, C₅₋₇cycloalkenylC₂₋₃alkynyl, C₁₋₄alkoxycarbonylamino, C₁₋₄alkanoylamino, C₁₋₄alkanoyl(N—C₁₋₄alkyl)amino, C₁₋₄alkanesulphonamido, benzenesulphonamido, aminosulphonyl, C₁₋₄alkylaminosulphonyl, di(C₁₋₄alkyl)aminosulphonyl, C₁₋₄alkoxycarbonyl, C₁₋₄alkanoyloxy, C₁₋₆alkanoyl, formylC₁₋₄alkyl, trifluoroC₁₋₃alkylsulphonyl, hydroxyiminoC₁₋₆alkyl, C₁₋₄alkoxyiminoC₁₋₆alkyl C₁₋₆alkylcarbamoylamino, oxazolyl, pyridyl, thiazolyl, pyrimidyl, pyrazinyl and pyridazinyl.

Where a ring nitrogen atom in A′ can be substituted without becoming quaternized, it is unsubstituted or substituted by C₁₋₄alkyl.

Particular substituents for ring carbon atoms in B′ include halo, amino, C₁₋₄alkylamino, di(C₁₋₄alkyl)amino, trifluoromethyl, nitro, hydroxy, C₁₋₆alkoxy, C₁₋₆alkyl, amino, C₁₋₄alkylamino, di(C₁₋₄alkyl)amino, cyano, —S(O)ₚC₁₋₆alkyl (p is 0, 1 or 2), carbamoyl, C₁₋₄alkylcarbamoyl and di(C₁₋₄alkyl)carbamoyl.

Where a ring nitrogen atom in B′ can be substituted without becoming quaternized, it is unsubstituted or substituted by C₁₋₄alkyl.

Particular substituents for optionally substituted groups in R^{e1}, R^{f} and R^{h} include those mentioned above for ring A′.

Particular substituents for carbon atoms in optionally substituted groups in R^{e1} include halo, hydroxy, C₁₋₄alkyl, nitro, cyano, amino, carboxy, trifluoromethyl, C₁₋₄alkoxy, C₃₋₇cycloalkyl, C₅₋₇cycloalkenyl, C₃₋₇cycloalkylC₁₋₃alkyl, C₅₋₇cycloalkenylC₁₋₃alkyl, C₃₋₇cycloalkylC₂₋₃alkenyl, C₅₋₇cycloalkenylC₂₋₃alkenyl and C₁₋₄alkoxycarbonyl. Particular substituents for optionally substituted groups in R^{f} include halo, trifluoromethyl, nitro, C₁₋₄alkyl, hydroxy, amino, cyano, amino, C₁₋₆alkoxy, S(O)ₚC₁₋₆alkyl (p is 0, 1 or 2), carbamoyl, C₁₋₄alkylcarbamoyl, di(C₁₋₄alkyl)carbamoyl, C₂₋₆alkenyl, C₂₋₆alkynyl, C₃₋₇cycloalkyl, C₅₋₇cycloalkenyl, C₃₋₇cycloalkylC₁₋₃alkyl, C₅₋₇cycloalkenylC₁₋₃alkyl, C₃₋₇cycloalkylC₂₋₃alkenyl, C₅₋₇cycloalkenylC₂₋₃alkenyl, C₁₋₄alkoxycarbonylamino, C₁₋₄alkanoylamino, C₁₋₄alkanoyl(N—C₁₋₄alkyl)amino, C₁₋₄alkanesulphonamido, benzenesulphonamido, aminosulphonyl, C₁₋₄alkylaminosulphonyl, di(C₁₋₄alkyl)aminosulphonyl, C₁₋₄alkoxycarbonyl, C₁₋₄alkanoyloxy, C₁₋₆alkanoyl, formylC₁₋₄alkyl, hydroxyiminoC₁₋₆alkyl, C₁₋₄alkoxyiminoC₁₋₆alkyl and C₁₋₆alkylcarbamoylamino.

The term alkyl when used herein includes straight chain and branched chain substituents for example methyl, ethyl, n-propyl, isopropyl, n-butyl and isobutyl and functional groups on alkyl chains may be anywhere on the chain, for example hydroxyiminoC₁₋₆alkyl includes 1-(hydroxyimino)propyl and 2-(hydroxyimino)propyl.

Examples of C₁₋₆alkoxycarbonyl are methoxycarbonyl, ethoxycarbonyl and t-butoxycarbonyl; examples of carboxyC₁₋₃alkyl are carboxymethyl, 2-carboxyethyl, 1-carboxyethyl and 3-carboxypropyl; examples of C₁₋₆alkoxycarbonylC₁₋₃alkyl are methoxycarbonylmethyl, ethoxycarbonylmethyl and methoxycarbonylethyl; examples of tetrazolylC₁₋₃alkyl are tetrazolylmethyl and 2-tetrazolylethyl; examples of C₁₋₄alkoxy are methoxy, ethoxy, propoxy and isopropoxy; examples of C₂₋₆alkenyl are vinyl and allyl; examples of C₂₋₆alkynyl are ethynyl and propynyl; examples of C₁₋₄alkanoyl are formyl, acetyl, propionyl and butyryl; examples of halo are fluoro, chloro, bromo and iodo; examples of C₁₋₄alkylamino are methylamino, ethylamino, propylamino and isopropylamino; examples of di(C₁₋₄alkyl)amino are dimethylamino, diethylamino and ethylmethylamino; examples of —S(O)ₚC₁₋₄alkyl are methylthio, methylsulphinyl and methylsulphonyl; examples of C₁₋₄alkylcarbamoyl are methylcarbamoyl and ethylcarbamoyl; examples of di(C₁₋₄alkyl)carbamoyl are dimethylcarbamoyl, diethylcarbamoyl and ethylmethylcarbamoyl; examples of C₁₋₆alkyl are methyl, ethyl, propyl and isopropyl; examples of C₃₋₇cycloalkyl are cyclopropyl, cyclobutyl and cyclohexyl; examples of C₃₋₇cycloalkylC₁₋₃alkyl are cyclopropylmethyl and cyclohexylmethyl; examples of C₃₋₇cycloalkylC₂₋₃alkenyl are cyclopropylethenyl and cyclopentylpropenyl, examples of C₃₋₇cycloalkylC₂₋₃alkynyl are cyclopropylethynyl and cyclopentylethynyl; examples of C₅₋₇alkenyl are cyclopentenyl and cyclohexenyl; examples of C₅₋₇cycloalkenylC₁₋₃alkyl are cyclopentenylmethyl and cyclohexenylmethyl; examples of C₅₋₇cycloalkenylC₂₋₃alkenyl are cyclohexenylethenyl and cycloheptenylethenyl; examples of C₅₋₇cycloalkenylC₂₋₃alkynyl are cyclopentenylethynyl and cyclohexenylethynyl; examples of C₁₋₄alkoxycarbonylamino are methoxycarbonylamino and ethoxycarbonylamino; examples of C₁₋₄alkanoylamino are acetamido and propionamido; examples of C₁₋₄alkanoyl(N—C₁₋₄alkyl)amino are N-methylacetamido and N-methylpropionamido; examples of C₁₋₄alkanesulphonamido are methanesulphonamido and ethanesulphonamido; examples of C₁₋₄alkylaminosulphonyl are methylaminosulphonyl and ethylaminosulphonyl; examples of di(C₁₋₄alkyl)aminosulphonyl are dimethylaminosulphonyl, diethylaminosulphonyl and ethylmethylaminosulphonyl; examples of C₁₋₄alkanoyloxy are acetyloxy and propionyloxy; examples of formylC₁₋₄alkyl are formylmethyl and 2-formylethyl; examples of hydroxyiminoC₁₋₆alkyl are hydroxyiminomethyl and 2-(hydroxyimino)ethyl; and examples of C₁₋₄alkoxyiminoC₁₋₆alkyl are methoxyiminomethyl, ethoxyiminomethyl and 2-(methoxyimino)ethyl.

Suitable ring systems of the formula (IIIA), (IIIB) or (IIIC) include 5-oxo-4,5-dihydro-1,2,4-oxadiazole-3-yl, 3-oxo-2,3-dihydro-1,2,4-oxadiazole-5-yl, 3-thioxo-2,3-dihydro-1,2,4-oxadiazole-5-yl, 5-oxo-4,5-dihydro-1,3,4-oxadiazole-2-yl, 5-oxo-4,5-dihydro-1,2,4-triazole-3-yl, 3-oxo-2,3-dihydroisoxazole-5-yl, 5-oxo-1,5-dihydroisoxazole-3-yl and 5-oxo-2,3-dihydropyrazol-3-yl.

Amino acid residues formed from R^{e} and R^{e1} together with the amide nitrogen to which they are attached and esters thereof include for example radicals of the formula —NH—CH(R^{g})—COOR^{h} wherein R^{g} is hydrogen, C₁₋₆alkyl, C₂₋₆alkenyl, C₂₋₆alkynyl, phenyl, phenylC₁₋₃alkyl, 5- or 6-membered heteroaryl or 5- or 6-membered heteroarylC₁₋₃alkyl and R^{h} is hydrogen or C₁₋₆alkyl, wherein alkyl, alkenyl, alkynyl, phenyl and heteroaryl groups are optionally substituted. Examples of substituents include those mentioned above for ring A′. In particular hydroxy.

When an alkenyl or alkynyl group is directly linked to the nitrogen of a primary or secondary amine it will be appreciated that the double or triple bond may not be in the 1-position. Similarly alkyl groups which are substituted by halo, hydroxy or an amine may not be substituted by these substituents in the 1-position when the alkyl group is directly linked to the nitrogen of a primary or secondary amine.

Preferably A′ is an optionally substituted:

phenyl, naphthyl, pyridyl, pyrazinyl, pyridazinyl, pyrimidyl, thienyl, thiazolyl, oxazolyl or thiadiazolyl having at least two adjacent ring carbon atoms;

More preferably A′ is optionally substituted:

phenyl, naphthyl, thiadiazolyl, thienyl, pyridyl or pyrimidyl.

Most preferably A′ is optionally substituted:

phenyl or thienyl.

In particular A′ is optionally substituted phenyl.

Preferably B′ is optionally substituted:

pyridyl, phenyl, thiazolyl, thienyl, pyridazinyl, thiadiazolyl, imidazolyl, pyrazinyl, pyrimidyl, or oxazolyl.

More preferably B′ is optionally substituted:

pyridyl, phenyl, thiazolyl, thienyl, pyridazinyl or oxazolyl.

Most preferably B′ is optionally substituted:

pyridyl, phenyl, thienyl, pyridazinyl or thiazolyl.

In particular B′ is optionally substituted:

pyrid-2,5-diyl, pyridazin-3,6-diyl, phen-1,4-diyl or thien-2,5-diyl.

Preferably D′ is optionally substituted: pyridyl, thienyl, thiazolyl, furyl or phenyl.

More preferably D′ is optionally substituted: thienyl, furyl or phenyl.

Most preferably D′ is optionally substituted phenyl.

Preferred optional substituents for ring carbon atoms in A′, are halo, nitro, trifluoromethyl, cyano, amino, C₁₋₆alkoxy, carbamoyl, C₁₋₆alkyl, C₃₋₇cycloalkyl, C₃₋₇cycloalkylC₁₋₃alkyl, C₃₋₇cycloalkylC₂₋₃alkenyl, C₅₋₇cycloalkenyl, C₅₋₇cycloalkenylC₁₋₃alkyl, C₅₋₇cycloalkenylC₂₋₃alkenyl, C₁₋₄alkylcarbamoyl, di(C₁₋₄alkyl)carbamoyl, C₁₋₄alkanoylamino, S(O)ₚC₁₋₆alkyl, C₁₋₄alkanesulphonamido, benzenesulphonamido, C₁₋₆alkanoyl, C₁₋₄alkoxyiminoC₁₋₄alkyl and hydroxyiminoC₁₋₄alkyl.

Most preferred optional substituents for ring carbon atoms in A′ are chloro, bromo and methanesulphonyl.

In particular A′ is substituted on a ring carbon atom by bromo.

Preferably, when A is a 6-membered ring, A′ is unsubstituted or substituted in the 4-position relative to the —O—CH(R¹³)— linking group.

Preferred optional substituents for ring carbon atoms of B′ are halo, amino, diC₁₋₄alkylamino, C₁₋₄alkylamino, trifluoromethyl, nitro, hydroxy, methyl, C₁₋₄alkyl, C₁₋₄alkoxy and cyano.

More preferred optional substituents for ring carbon atoms of B′ are fluoro, chloro, bromo, trifluoromethyl, hydroxy, methyl, methoxy and cyano.

Preferably D′ is optionally substituted by 1 or 2 substituents selected from halo, trifluoromethyl, nitro, hydroxy, amino, C₁₋₄alkylamino, di(C₁₋₄alkyl)amino, cyano, C₁₋₆alkoxy, —S(O)ₚC₁₋₄alkyl (p is 0, 1 or 2), C₁₋₄alkanoyl, C₁₋₆alkyl, C₃₋₇cycloalkyl, C₃₋₇cycloalkylC₁₋₃alkyl, C₃₋₇cycloalkylC₂₋₃alkenyl, C₅₋₇cycloalkenyl, C₅₋₇cycloalkenylC₁₋₃alkyl, C₅₋₇cycloalkenylC₂₋₃alkenyl, wherein C₃₋₇cycloalkyl, C₅₋₇cycloalkenyl, C₁₋₆alkyl and C₁₋₆alkyloxy are optionally substituted by trifluoromethyl, hydroxy, halo, nitro, cyano or amino.

Most preferred optional substituents for D′ include halo, nitro, hydroxy, cyano, C₁₋₆alkyl, amino, C₁₋₆alkoxy or carbamoyl. Most preferably D′ is unsubstituted.

Preferably A′ is unsubstituted or substituted by one substituent.

Preferably B′ is unsubstituted or substituted by one substituent.

Preferably R¹⁰ is carboxy, carbamoyl, tetrazolyl or of the formula —CONR^{e}R^{e1} or —CONHSO₂R^{f}.

Preferably, R^{e1} is hydrogen, hydroxy or optionally substituted: C₁₋₆alkyl, C₂₋₆alkenyl, C₂₋₆alkynyl, cyclopropylC₁₋₄alkyl, cyclobutylC₁₋₄alkyl, cyclopentylC₁₋₄alkyl, cyclohexylC₁₋₄alkyl, pyridylC₁₋₄alkyl, pyrimidylC₁₋₄alkyl, pyrazinylC₁₋₄alkyl, furylC₁₋₄alkyl, pyridazinylC₁₋₄alkyl, tetrazolylC₁₋₄alkyl, pyrrolidinylC₁₋₄alkyl, morpholinylC₁₋₄alkyl, imidazoliumC₁₋₄alkyl, N-methylimidazoliumC₁₋₄alkyl, pyridiniumC₁₋₄alkyl, pyridyl, pyrimidyl, pyrazinyl, pyridazinyl, N-methylpyrimidinium, N-methylimidazolyl, pyridinium, pyrimidinium, tetrazolyl, phenyl, cyclopropyl, cyclobutyl, cyclopentyl, cyclohexyl,

cyclopentenyl, cyclohexenyl, cycloheptenyl, cyclopentenylC₁₋₄alkyl, cyclohexenylC₁₋₄alkyl or cycloheptenylC₁₋₄alkyl.

More preferably aspect R^{e1} is hydrogen, C₁₋₆alkyl (optionally substituted by halo, hydroxy, nitro, cyano, amino, carboxy, C₁₋₄alkoxycarbonyl), pyridylC₁₋₄alkyl, pyrimidylC₁₋₄alkyl, pyrazinylC₁₋₄alkyl, furylC₁₋₄alkyl, pyridazinylC₁₋₄alkyl, tetrazolylC₁₋₄alkyl, or C₂₋₆alkenyl.

Most preferably R^{e1} is C₁₋₄alkyl (optionally substituted by one or two substituents selected from hydroxy, carboxy and C₁₋₄alkoxycarbonyl), pyridylC₁₋₄alkyl and furylC₁₋₄alkyl.

Preferably —C₁₋₃alkylCONR^{e}R^{e1} is —CH₂CONR^{e}R^{e1}.

Preferably —C₁₋₃alkylCONHSO₂R^{f} is —CH₂CONHSO₂R^{f}.

Preferably —C₁₋₃alkylCONR^{e}NR^{g}R^{h} is —CH₂CONR^{e}NR^{g}Rh^{d}.

Preferably R^{f} is optionally substituted: C₁₋₆alkyl,

C₃₋₇cycloalkyl, C₃₋₇cycloalkylC₁₋₃alkyl, C₃₋₇cycloalkylC₂₋₃alkenyl, C₅₋₇cycloalkenyl, C₅₋₇cycloalkenylC₁₋₃alkyl, C₅₋₇cycloalkenylC₂₋₃alkenyl, 5- or 6-membered heteroarylC₁₋₃alkyl, 5- or 6-membered saturated or partially saturated heterocyclylC₁₋₃alkyl, phenylC₁₋₃alkyl, phenyl, 5- or 6-membered heteroaryl or 5- or 6-membered saturated or partially saturated heterocyclyl.

More preferably R^{f} is C₁₋₄alkyl (optionally substituted by hydroxy, nitro, cyano, amino, C₁₋₁₄alkylamino, di-C₁₋₄alkylamino, C₁₋₄alkanoylamino, C₁₋₄alkyl-N—C₁₋₄alkanoylamino, carbamoyl, C₁₋₄alkylcarbamoyl, di-C₁₋₄alkanoylcarbamoyl, halo, C₁₋₄alkoxy) or optionally substituted phenylC₁₋₃alkyl, pyridylC₁₋₃alkyl, phenyl, thienyl, thiadiazolyl, oxazolyl, isoxazolyl, pyrazolyl or 1,1-dioxidotetrahydrothienyl.

Most preferably R^{f} is C₁₋₄alkyl, hydroxyC₁₋₄alkyl, C₁₋₄alkoxyC₁₋₄alkyl, phenyl (optionally substituted by halo, cyano, nitro, carbamoyl, C₁₋₄alkylcarbamoyl, di-C₁₋₄alkylcarbamoyl, hydroxy, amino, C₁₋₄alkanoylamino, N-C₁₋₄alkanoyl —N—C₁₋₄alkylamino, C₁₋₄alkylamino or di-(C₁₋₄alkyl)amino), benzyl (optionally substituted by halo, cyano, nitro, carbamoyl, C₁₋₄alkylcarbamoyl, di-C₁₋₄alkylcarbamoyl, hydroxy, amino, C₁₋₄alkanoylamino, N-C₁₋₄alkanoyl-N-C₁₋₄alkylamino, C₁₋₄alkylamino or di-(C₁₋₄alkyl)amino), thiadiazolyl (optionally substituted by C₁₋₄alkanoylamino, amino, C₁₋₄alkylamino or di-C₁₋₄alkylamino), thienyl (optionally substituted by halo or pyridyl), isoxazolyl (optionally substituted by C₁₋₄alkyl or halo), pyrazolyl (optionally substituted by C₁₋₄alkyl or halo) or 1,1-dioxidotetrahydro-2-thienyl.

Preferably R^{g} is hydrogen and R^{h} is 5- or 6-membered heteroaryl or R^{g} and R^{h}, together with the nitrogen atom to which they are attached, form a 5- or 6-membered saturated or partially saturated heterocyclic ring.

More preferably R^{g} is hydrogen and R^{h} is pyridyl or R^{g} and R^{h}, together with the nitrogen atom to which they are attached, form morpholino.

In one aspect R¹⁰ is carboxy, carbamoyl or tetrazolyl or R¹⁰ is of the formula —CONR^{e} R^{e1} wherein R^{e} is hydrogen or C₁₋₆alkyl and R^{e1} is C₁₋₆alkyl (optionally substituted by hydroxy), C₂₋₆alkenyl, 1-morpholinyl, 1-piperidinyl, 1-pyrrolidinyl, pyridylC₁₋₃alkyl or R¹⁰ is of the formula —CONHSO₂R^{f} wherein R^{f} is C₁₋₆alkyl or phenyl.

In another aspect, R¹¹⁰ is carboxy, tetrazolyl or of the formula —CONR^{e} R^{e1} wherein R^{e} is hydrogen and R^{e1} is C₁₋₆alkyl (optionally substituted by hydroxy) or pyridylmethyl, or R¹⁰ is of the formula —CONHSO₂R^{f} wherein R^{f} is C₁₋₆alkyl or phenyl.

Most preferably R¹⁰ is carboxy.

More preferably R¹¹ is hydrogen, methyl, ethyl, cyclopropylmethyl, 2-fluoroethyl, 2,2-difluoroethyl, 2,2,2-trifluoroethyl, cyanomethyl, allyl or 2-propynyl.

Most preferably R¹¹ is ethyl, allyl or 2-propynyl.

In particular R¹¹ is ethyl.

In one aspect R¹¹ is hydrogen, C₁₋₆alkyl (optionally substituted by hydroxy, cyano or trifluoromethyl), C₂₋₆alkenyl, C₂₋₆alkynyl, phenylC₁₃alkyl or pyridylC₁₋₃alkyl;

Preferably R¹² is hydrogen, methyl or ethyl.

Preferably R¹³ is hydrogen, methyl or ethyl.

Most preferably R¹² is hydrogen or methyl.

Most preferably R¹³ is hydrogen.

Compounds of Formula III, for use in this invention are described in WO 96/03380 which is incorporated by reference herein.

A preferred compound of formula III for use in this invention is:

6-[N-(2-benzyloxy-5-bromobenzyl)-N-ethylamino]pyridazine-3-carboxylic acid (described as Example 15 in International Patent Application WO 96/03380);

Yet further compounds useful in this invention include compounds of the formula (IV) and (V).

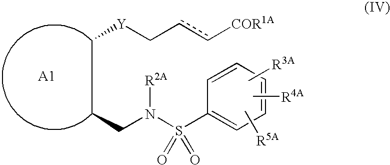

wherein, in formula IV, A1

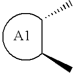

denotes a group represented by the following formulae:

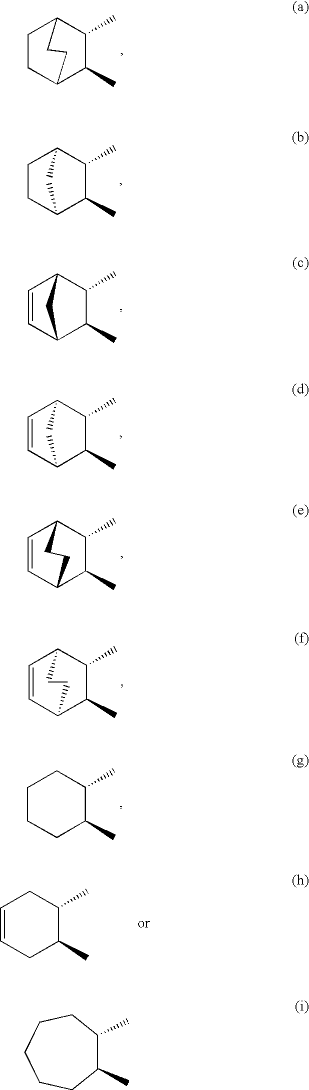

R^{1A} denotes a hydroxy, alkoxy group of C₁-C₄ or a group represented by general formula NR^{6A}R^{7A}, wherein, R^{6A} and R^{7A} each independently represent hydrogen atom or alkyl group of C₁-C₄,

R^{2A} denotes hydrogen atom or alkyl group of C₁-C₄,

R^{3A} and R^{4A} denotes alkyl group of C₁-C₄, halogen atom or trifluoromethyl group,

R^{5A} denotes hydrogen atom, alkyl group of C₁-C₄, halogen atom or trifluoromethyl group,

Y denotes cis-vinylene or trans-vinylene,

and the symbol [symbol] denotes single bond or double bond.

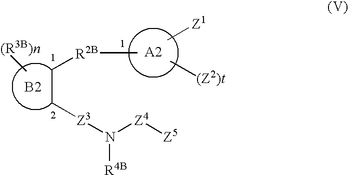

In formula V,

Ring A2 and ring B2 each independently denote carbon-ring of C₅-C₁₋₅ or 5-7 membered heterocyclic ring having 1 or 2 oxygen, sulfur or nitrogen atom,

Z¹ comprises a group represented by

(1) —COR^{1B}.

(2) —C₁-C₄ alkylene-COR^{1B}.

(3) —CH═CH—COR^{1B}.

(4) —C≡C—COR^{1B}.

(5) —CO—C₁-C₃ alkylene-COR^{1B}

In the formula, R^{1B} denotes a hydroxy group, C₁-C₄ alkoxy or a group represented by formula NR^{6B}R^{7B}, wherein, R^{6B} and R^{7B} independently denotes hydrogen or C₁-C₄ alkyl; or

(6) —C₁-C₅ alkylene-OH,

Z² denotes hydrogen atom, C₁-C₄ alkyl, C₁-C₄ alkoxy, nitro, halogen, trifluoromethyl, trifluoro methoxy, hydroxy group or a group represented by formula COR^{1B}, wherein, R^{1B} has the same aforesaid meaning),

Z³ represents single bond or C₁-C₄ alkylene,

Z⁴ represents SO₂ or CO,

Z⁵ denotes

(1) C₁-C₈ alkyl, C₂-C₈ alkenyl or C₂-C₈ alkynyl,

(2) phenyl, C₃-C₇ cycloalkyl or 5-7 membered hetero-ring having 1 or 2 oxygen, sulfur or nitrogen atoms,

(3) phenyl or C₃-C₇ cycloalkyl-substituted C₁-C₄ alkyl, C₂-C₄ alkenyl or C₂-C₄ alkynyl,

In aforesaid (2) and (3), phenyl, C₃-C₇ cycloalkyl, 5-7 membered heterocyclic ring having 1 or 2 oxygen, sulfur or nitrogen atoms, may be substituted with 1-5 R^{5B} groups wherein a plurality of R^{5B} groups independently denote hydrogen atom, C₁-C₆ alkyl, C₁-C₆ alkoxy, C₁-C₆ alkylthio, nitro, halogen, trifluoromethyl, trifluoro methoxy or hydroxy group)),

R^{2B} denotes CONR^{8B}, NR^{8B}CO,

CONR^{8B}—C₁-C₄ alkylene, C₁-C₄ alkylene-CONR^{8B}, NR^{8B}CO—C₁-C₄ alkylene, C₁-C₄ alkylene-NR^{8B}CO, C₁-C₃ alkylene-CONR^{8B}C₁-C₃ alkylene, C₁-C₃ alkylene-NR^{8B}CO—C₁-C₃ alkylene (in each formula, R^{8B} denotes hydrogen atom or C₁-C₄ alkyl), O, S, NZ⁶ (wherein, Z⁶ denotes hydrogen atom or C₁-C₄ alkyl), Z⁷-C₁-C₄ alkylene, C₁-C₄ alkylene-Z⁷, a group represented by C₁-C₃ alkylene-Z⁷-C₁-C₃ alkylene (wherein, Z⁷ denotes O, S or NZ⁶ (wherein, Z⁶ has the same aforesaid meaning)), CO, CO—C₁-C₄ alkylene, C₁-C₄ alkylene-CO, C₁-C₃ alkylene-CO—C₁-C₃ alkylene, C₂-C₄ alkylene, C₂-C₄ alkenylene or C₂-C₄ alkynylene,

R^{3B} denotes hydrogen atom, C₁-C₆ alkyl, C₁-C₆, alkoxy, C₁-C₆ alkylthio, nitro, halogen, trifluoromethyl, trifluoro methoxy, hydroxy group or hydroxymethyl,

R^{3B} denotes

(1) hydrogen atom,

(2) C₁-C₈ alkyl, C₂-C₈ alkenyl or C₂-C₈ alkynyl,

(3) 1 or 2 COOZ⁸, CONZ⁹Z¹⁰, OZ⁸ group (in each group, Z⁸, Z⁹, Z¹⁰ each independently denote hydrogen atom or C₁-C₄ alkyl) and C₁-C₆ alkyl substituted by the group selected from the group comprising C₁-C₄ alkoxy-C₁-C₄ alkoxy,

(4) C₃-C₇ cycloalkyl,

(5) phenyl or C₃-C₇ cycloalkyl-substituted C₁-C₄ alkyl, C₂-C₄ alkenyl or C₂-C₄ alkynyl.

In aforesaid (4) and (5), phenyl, C₃-C₇ cycloalkyl may be substituted with 1-5 R^{5B} groups wherein R^{5B} has the same aforesaid meaning,

n and t respectively independently represent an integer of 1-4.

Wherein,

(1) R² and R³ each bond only at 1 and 2 position of ring B2

(2) when ring A2 represents a benzene ring

and (Z²)ₜ does not represent COR^{1B}, Z¹ is bonded only at 3 or 4 positions of the benzene ring.

These compounds of formula IV and formula V for use in the present invention are described in WO 00/69465 which is incorporated by reference herein. Preferred compounds of formula IV and formula V for use in the present invention are:

6-[(2S,3 S)-3-(4-chloro-2-methylphenylsulfonylaminomethyl)-bicyclo[2.2.2]octan-2-yl]-5Z-hexenoic acid (described as compound A in International Patent application WO 00/69465);

4-[2-[N-isobutyl-N-(2-furylsulfonyl)amino]-5-trifluoromethylphenoxymethyl]cinnamic acid (described as compound B in International Patent application WO 0.00/69465);

or a pharmaceutically acceptable salt or an in vivo hydrolysable ester thereof, or a cyclodextrin inclusion complex, or a sulfonamide or carboxamide derivative thereof.

The EP1 antagonist properties of a compound may be demonstrated using test procedures described in U.S. Pat. No. 5,994,353, col. 13, lines 25-65. The contents of aforesaid U.S. Pat. No. 5,994,353 are hereby incorporated by reference.

Hence, there is provided herein a method of reducing uric acid levels in a warm-blooded animal, such as a human being requiring such reduction, which comprises administering to said animal a therapeutically effective amount of the compound described above, preferably the compounds of formula I, formula II, formula III, formula IV or formula V above.

According to a further feature of the invention, there is provided a method of treating hyperuricemia in a warm blooded animal such as a human being which comprises administering to said animal a therapeutically effective amount of the compound described above, preferably a compound of formula I, formula II, formula III, formula Iv or formula V above, or a pharmaceutically acceptable salt or an in vivo hydrolysable ester thereof.

In another aspect of this invention, there is provided a method of treating or preventing a disease caused by hyperuricemia in a warm-blooded animal, such as a human, comprising administering to the animal a therapeutically effective amount of the compound described above, preferably a compound of formula I, formula II, formula III, formula IV or formula V above, or a pharmaceutically acceptable salt or an in vivo hydrolysable ester thereof.

The diseases that may be treated comprise hyperuricemia, gout, gouty arthritis, trophi, hyperuricemia associated with a congenital abnormality in the metabolism of xanthine oxidase, urate nephropathy incident to hyperuricemia, hyperuricemia incident to cytotoxic chemotherapy and hyperuricemia incident to radiation therapy.

According to a further aspect of the invention there is provided the use of a compound described above, preferably a compound of formula I, formula II, formula III, formula IV or formula V above, or a pharmaceutically acceptable salt or an in vivo hydrolysable ester thereof, for the manufacture of a medicament for use in:

1) reducing uric acid levels in a warm-blooded animal such as a human;

2) treating hyperuricemia; or

3) treating or preventing a disease caused by hyperuricemia in a warm-blooded animal.

In another aspect of the invention there is provided the use of a compound that is an EP1 antagonist or a pharmaceutically acceptable salt or an in vivo hydrolysable ester thereof, for:

1) reducing uric acid levels in a warm-blooded animal such as a human;

2) treating hyperuricemia; or

3) treating or preventing a disease caused by hyperuricemia in a warm-blooded animal.

Preferred compounds of the invention are those of formula I, formula II, formula III, formula IV and formula V above, or a pharmaceutically acceptable salt or an in vivo hydrolysable ester thereof.

A particularly preferred compound is the compound: N-propanesulfonyl-6-[N-(5-bromo-2-(cyclopropylmethoxy)benzyl)-N-ethylamino]pyridazine-3-carboxamide, or a pharmaceutically acceptable salt or an in vivo hydrolysable ester thereof.

It will be understood that when compounds of the present invention contain a chiral center, the compounds of the invention may exist in, and be isolated in, optically active or racemic form. The invention includes any optically active or racemic form of a compound of the present invention which possesses uric acid reducing properties. The synthesis of optically active forms may be carried out by standard techniques of organic chemistry well known in the art, for example by, resolution of a racemic form, by synthesis from optically active starting materials or by asymmetric synthesis. It will also be appreciated that certain compounds of the present invention may exist as geometrical isomers. The invention includes any geometrical isomer of a compound of the present invention which possesses uric acid lowering properties.

It will also be understood that certain compounds of the present invention may exist in solvated, for example hydrated, as well as unsolvated forms. It is to be understood that the present invention encompasses all such solvated forms which possess the property of reducing uric acid levels.

It will further be understood that the present invention encompasses tautomers of the compounds of the formula I, formula II, formula III, formula IV and formula V.

Further, it will be understood that compounds of the present invention will include pharmaceutically acceptable salts and ester derivatives which are hydrolysable in vivo.

Generally, pharmaceutically acceptable salts of compounds of the present invention may be obtained using standard procedures well known in the art, for example by reacting a sufficiently basic compound (or its ester) with a suitable acid to afford a physiologically acceptable anion. It may also be possible to make a corresponding alkali metal (e.g. sodium, potassium, or lithium) or alkaline earth metal (e.g. calcium) salt by treating a compound of the present invention having a suitably acidic proton, such as a carboxylic acid (and in some cases the ester) with one equivalent of an alkali metal or alkaline earth metal hydroxide or alkoxide (e.g. the ethoxide or methoxide), or a suitably basic organic amine (such as choline or meglumine) in aqueous medium followed by conventional purification techniques.

An in vivo hydrolysable ester of a compound of the invention containing a carboxy group is, for example, a pharmaceutically acceptable ester which is hydrolysed in the human or animal body to produce the parent acid, for example, a pharmaceutically acceptable ester formed with a C₁₋₆alcohol such as methanol, ethanol, ethylene glycol, propanol or butanol, or with a phenol or benzyl alcohol such as phenol or benzyl alcohol or a substituted or multiply substituted phenol or benzyl alcohol wherein the substituent is, for example, a halo (such as fluoro or chloro), C₁₋₄alkyl (such as methyl) or C₁₋₄alkoxy (such as ethoxy) group. The term also includes α-acyloxyalkyl esters and related compounds which break down to give the parent hydroxy group. Examples of α-acyloxyalkyl esters include acetoxymethoxycarbonyl and 2,2-dimethylpropionyloxymethoxycarbonyl.

In use for reducing uric acid levels in a warm-blooded animal such as a human, an EP1 antagonist will generally be administered in the form of a conventional pharmaceutical composition, for example, as may be described in the relevant published European, US or International patent applications referred to above, and generally the composition may be in a form suitable for oral or sublingual administration, for example as a tablet or capsule, for parenteral injection (including intravenous, subcutaneous, intramuscular, intravascular or infusion) for example as a sterile solution, suspension or emulsion, for topical administration for example as an ointment or cream or for rectal administration for example as a suppository. In general the above compositions may be prepared in a conventional manner using conventional carriers. The compositions of the present invention are advantageously presented in unit dosage form. Subsequent to administration, one of ordinary skill in the art may determine if a particular compound reduces uric acid levels by analysing blood levels of uric acid using standard blood analysis procedures.

A therapeutically effective amount for the practice of the present invention may be determined, by the use of known criteria within the context of the disease which is being treated or which is being prevented, by one of ordinary skill in the art. A suitable therapeutically effective dose of the compound will normally be administered to a warm-blooded animal within the range of 5-5000 mg per square meter body area of the animal, i.e., approximately 0.1-100 mg/kg.

Therefore, a further feature of the invention is a pharmaceutical composition which comprises an EP1 antagonist, or a pharmaceutically acceptable salt or an in vivo hydrolysable ester thereof, in association with a pharmaceutically acceptable carrier for reducing uric acid levels in a warm-blooded animal such as a human. In practicing this invention a reduction in uric acid blood levels of about 29-42% have been observed.

Additionally, there is provided a pharmaceutical composition which comprises an EP1 antagonist, or a pharmaceutically acceptable salt or an in vivo hydrolysable ester thereof, in association with a pharmaceutically acceptable carrier for the treatment of hyperuricemia.

Treatment of a disease within the context of the present invention means to administer a an effective amount of a compound described for use in this invention, to mitigate either a pre-existing disease state, acute or chronic, or a recurring condition. This definition also encompasses prophylactic therapies for prevention of recurring conditions and continued treatment of chronic disorders. Treatment further means to alleviate associated symptoms and signs of hyperuricemia. Examples of these symptoms include the precipitation of solid crystalline deposits of uricacid in soft tissues. These deposits may be in joint tissue associated with gout or gouty arthritis. The deposits may be in the microtubules of the kidney and associated with renal calculi, hyperuricemic nephropathy, urinary tract infections and renal insufficiency. The deposits may occur in other soft tissues as trophi. Symptoms of infantile hyperuricemia may involve growth and motor retardation and sensorineural deafness. The practice of the present invention, either as a single therapeutic agent, or in combination with a conventional therapy, may be for treatment of a pre-existing condition of hyperuricemia or a disease associated with hyperuricemia such as gout, by reducing uric acid levels.

Prevention of hyperuricemia and diseases associated with hyperuricemia means control of uric acid levels by prophylactic administration of compounds of the present invention in order to prevent hyperuricemia and to prevent diseases associated with hyperuricemia.

Often, diseases associated with hyperuricemia are recurring conditions. It will be understood that the risk of occurrence or recurrence of diseases associated with hyperuricemia, or elevated levels of uric acid, is directly related to the degree of elevation of uric acid levels. Hence, compounds of the present invention, either as a single therapeutic agent, or in combination with a conventional therapy, may be used in the prevention of recurring episodes of hyperuricemia or diseases associated with hyperuricemia, for example, by prophylactic dosing to control uric acid levels and prevent the hyperuricemia which is the underlying cause of diseases such as gout and gouty arthritis.

Accordingly, there is provided herein a method of lowering uric acid levels useful in treatment of hyperuricemia and diseases associated with hyperuricemia, including, but not limited to, gout, gouty arthritis, trophi, hyperuricemia associated with a congenital abnormality in the metabolism of xanthine oxidase, urate nephropathy incident to hyperuricemia, or hyperuricemia incident to therapeutic use of cytotoxic chemotherapy.

In addition, there is also provided a method of preventing subsequent elevated uric acid levels as prophylactic therapy to prevent the recurrence of hyperuricemia and diseases associated with hyperuricemia, including, but not limited to, gout, gouty arthritis, trophi, hyperuricemia associated with a congenital abnormality in the metabolism of xanthine oxidase, urate nephropathy incident to hyperuricemia, or hyperuricemia incident to therapeutic use of cytotoxic chemotherapy, comprising prophylactic therapy or combination therapy of uric acid lowering agent with said cytotoxic agents or radiation therapy.

Compounds of the present invention may additionally be used as preventative therapy involving a combination therapy comprising co-administration of compounds or pharmaceutical compositions of the present invention with cytotoxic agents or radiation therapy for the treatment of cancers, including lymphomas, leukemia, and solid tumors, said co-administration being for the treatment or prevention of hyperuricemia associated with the administration of said cytotoxic agents or radiation therapy.

Accordingly, there is provided herein a method of preventing elevation of uric acid levels, associated with administration of cytotoxic agents or radiation therapy involved in the treatment of cancers, including lymphomas, leukemia, and solid tumors, comprising coadministration of an EP1 antagonist with said cytotoxic or radiation therapy.

Additionally, there is provided herein a method of preventing elevation of uric acid levels, associated with administration of cytotoxic agents or radiation therapy involved in the treatment of cancers, including lymphomas, leukemia, and solid tumors, comprising coadministration of a compound of formula I, formula II, formula III, formula IV or formula V described above with said cytotoxic or radiation therapy.

EP1 antagonists of the present invention may be used to reduce uric acid levels in single therapeutic agent therapy or in combination therapy. Combination therapy may involve current conventional therapeutic agents used in the management of hyperuricemia. Such combination therapy may involve concomitant use of a conventional therapeutic uricosuric agent such as probenecid or sulfinpyrazone, or in combination with agents known to inhibit the biosynthesis of uric acid, such as allopurinol. In addition, therapy may be effected in combination with such conventional therapies as NSAIDS, such as indomethacin, ketorolac, acetylsalicylic acid, ibuprofen, sulindac, tolmetin and piroxicam; or colchicine; or corticosteroids such as methylprednisolone which address the inflammation associated with crystalline deposits of uric acid in conditions such as gout or gouty arthritis. Combination therapy may also involve an opiate. Combination therapies may also employ strategies such as hydration, osmotic diuresis and urinary alkalinization with agents such as sodium bicarbonate, in addition to administration of compounds of this invention. In addition, combination therapies incorporating the present invention may involve use of any therapeutically effective combination of the aforementioned therapeutic agents or treatments.

The following Example is illustrative only, and is not meant to limit this invention in any manner.

### EXAMPLE 1

Protocols for Dosing and Administration:

Test subjects were orally administered tablets containing N-propanesulfonyl-6-[N-(5-bromo-2-(cyclopropylmethoxy)benzyl)-N-ethylamino]pyridazine-3-carboxamide, or a matching placebo was administered orally as tablets, with 200 mL of distilled water. The subjects were directed to remain semi-recumbent for six hours after dosing. A 4.9 mL sample of blood was taken into a tube containing lithium heparin anticoagulant, and plasma was separated by centrifugation for the clinical chemistry analysis at the pretrial medical examinations, pre-dose, 6 hours and 24 hours after dosing on each trial day. Concentrations of the uric acid were determined by standard analytical techniques. The data gathered on compounds of the present invention is depicted in Table 1 below.

TABLE 1
Clinical data for reduction of uric acid levels in human volunteers.

| | | Dose in mg. | | | | | | | | | |
|---|---|---|---|---|---|---|---|---|---|---|---|
| Time | data | 1 | 5 | 25 | 100 | 200 | 400 | 800 | placebo | 1600 | placebO |
| before | mea | .284 | .272 | .254 | .317 | .314 | .343 | .366 | .3126 | .293 | .3053 |
| dose | SD | .018 | .047 | .061 | .049 | .082 | .057 | .066 | .0563 | .065 | .0880 |
| | Min. | .259 | .228 | .174 | .276 | .234 | .299 | .266 | .239 | .193 | .195 |
| | Max. | .299 | .328 | .321 | .375 | .429 | .424 | .423 | .429 | .357 | .421 |
| | n | 4 | 4 | 4 | 4 | 4 | 4 | 4 | 14 | 6 | 6 |
| 24 hr. | Mea | 280 | 245 | 237 | 286 | 268 | 242 | 237 | 2976 | 168 | 2837 |
| after | SD | .008 | .041 | .063 | .031 | .056 | .042 | .047 | .0474 | .038 | .0724 |
| dose | Min. | .269 | .205 | .157 | .250 | .212 | .195 | .186 | .242 | .123 | .184 |
| | Max. | .290 | .287 | .294 | .318 | .346 | .289 | .299 | .426 | .217 | .361 |
| | n | 4 | 4 | 4 | 4 | 4 | 4 | 4 | 14 | 6 | 6 |

| | | | | | | | | | | |
|---|---|---|---|---|---|---|---|---|---|---|
| % at 24 hrs. | 98.8 | 90.1 | 93.2 | 90.0 | 85.2 | 70.6 | 66.7 | 95.2 | 57.6 | 92.9 |

The listed percentages represent mean blood level of uric acid at 24 hours after administration of the test substance (active compound or placebo) as a percentage of the baseline uric acid level measured prior to administration. The data demonstrates substantial reduction in serum levels of uric acid in a well-defined dose-dependent manner. In particular at a dose of 400 mg the mean uric acid blood level is reduced by 29%. Doses of 800 mg and 1600 mg likewise reduce blood levels of uric acid by 33% and 42% respectively.

1. A method of reducing uric acid levels in a warm-blooded animal, comprising administering to said animal an effective amount of an EP1 antagonist, or a pharmaceutically acceptable salt or an in vivo hydrolysable ester of said antagonist.

2. A method of treating hyperuricemia in a warm blooded animal comprising administering to said animal a therapeutically effective amount of an EP1 antagonist, or a pharmaceutically acceptable salt or an in vivo hydrolysable ester of said antagonist.

3. A method of treating or preventing a disease caused by hyperuricemia in a warm-blooded animal comprising administering to said animal a therapeutically effective amount of an EP1 antagonist or a pharmaceutically acceptable salt or an in vivo hydrolysable ester of said antagonist.

4. The method as recited in claim 3 wherein the disease is selected from the group consisting of hyperuricemia, gout, gouty arthritis, trophi, hyperuricemia associated with a congenital abnormality in the metabolism of xanthine oxidase, urate nephropathy incident to hyperuricemia, hyperuricemia incident to cytotoxic chemotherapy or hyperuricemia incident to radiation therapy.

5. A method of reducing uric acid levels in a warm-blooded animal comprising administering to said animal a therapeutically effective amount of a compound of the formula I or formula II: 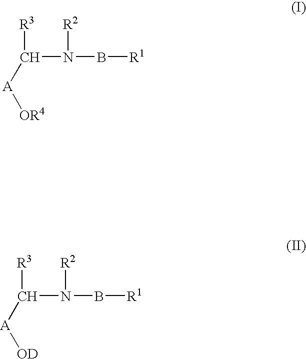
wherein:
A is an optionally substituted:
phenyl, naphthyl, pyridyl, pyrazinyl, pyridazinyl, pyrimidyl, thienyl, thiazolyl, oxazolyl or thiadiazolyl having at least two adjacent ring carbon atoms;
provided that the —CH(R³)N(R²)B—R¹ and —OR⁴ groups (formula I) or OD groups (formula II) are positioned in a 1,2 relationship to one another on ring carbon atoms and the ring atom positioned ortho to the OR⁴ linking group of formula I or the OD group of formula II (and therefore in the 3-position relative to the —CHR³NR²— linking group) is not substituted;
B is an optionally substituted:
phenyl, pyridyl, thiazolyl, oxazolyl, thienyl, thiadiazolyl, imidazolyl, pyrazinyl, pyridazinyl or pyrimidyl;
R¹ is positioned on ring B in a 1, 3 or 1,4 relationship with the —CHR³NR²— linking group and is carboxy, carboxyC₁₋₃alkyl, tetrazolyl, tetrazolylC₁₋₃alkyl, tetronic acid, hydroxamic acid, sulphonic acid, or R¹ is of the formula —CONR^{a}R^{a1} wherein R^{a} is hydrogen or C₁₋₆alkyl and R^{a1} is hydrogen, C₁₋₆alkyl (optionally substituted by halo, amino, C₄alkylamino, di-C₁₋₄ alkylamino, hydroxy, nitro, cyano, trifluoromethyl, C₁₋₄alkoxy or C₁₋₄alkoxycarbonyl), C₂₋₆alkenyl (provided the double bond in not in the 1-position), C₂₋₆alkynyl (provided the triple bond is not in the 1-position), carboxyphenyl, 5- or 6-membered heterocyclylC₁₋₃alkyl, 5- or 6-membered heteroarylC₁₋₃alkyl, 5- or 6-membered heterocyclyl, or 5- or 6-membered heteroaryl or R^{a} and R^{a1} together with the amide nitrogen to which they are attached (NR^{a}R^{a1}) form an amino acid residue or ester thereof, or R¹ is of the formula —CONHSO₂R^{b} wherein R^{b} is C₁₋₆alkyl (optionally substituted by halo, hydroxy, nitro, cyano, trifluoromethyl, C₁₋₄alkoxy, amino, C₁₋₄alkylamino, di-C₁₋₄alkylamino or C₁₋₄alkoxycarbonyl), C₂₋₆alkenyl (provided the double bond is not in the 1-position), C₂₋₆alkynyl (provided the triple bond is not in the 1-position), 5- or 6-membered heterocyclylC₁₋₃alkyl, 5- or 6-membered heteroarylC₁₋₃alkyl, phenylC₁₋₃alkyl, 5- or 6-membered heterocyclyl, 5- or 6-membered heteroaryl or phenyl; wherein any heterocyclyl or heteroaryl group in R^{a1} is optionally substituted by halo, hydroxy, nitro, cyano, trifluoromethyl, C₁₋₄alkoxy or C₁₋₄alkoxycarbonyl and any phenyl, heterocyclyl or heteroaryl group in R^{b} is optionally substituted by halo, trifluoromethyl, nitro, hydroxy, amino, cyano, C₁₋₆alkoxy, C₁₋₆alkylS(O)ₚ-(p is 0, 1 or 2), C₁₋₆alkyl carbamoyl, C₁₋₄alkylcarbamoyl, di(C₁₋₄alkyl)carbamoyl, C₂₋₆alkenyl, C₂₋₆alkynyl, C₁₋₄alkoxycarbonylamino, C₁₋₄alkanoylamino, C₁₋₄alkanoyl(N—C₄alkyl)amino, C₁₋₄alkanesulphonamido, benzenesulphonamido, aminosulphonyl, C₁₋₄alkylaminosulphonyl, di(C₁₋₄alkyl)aminosulphonyl, C₁₋₄alkoxycarbonyl, C₁₋₄alkanoyloxy, C₁₋₆alkanoyl, formylC₁₋₄alkyl, hydroxyiminoC₁₋₆alkyl, C₁₋₄alkoxyiminoC₁₋₆alkyl or C₁₋₆alkylcarbamoylamino; or R¹ is of the formula —SO₂N(R^{c})R^{c1} wherein R^{c} is hydrogen or C₁₋₄alkyl and R¹ is hydrogen or C₁₋₄alkyl;
or R¹ is of the formula (IA), (IB) or (IC): 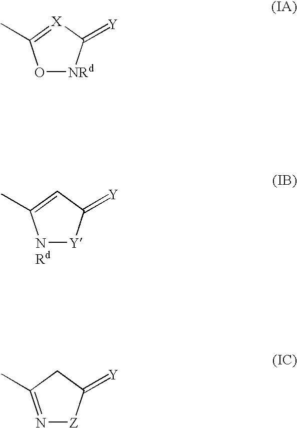
wherein X is CH or nitrogen, Y is oxygen or sulphur, Y′ is oxygen or NR^{d} and Z is CH₂, NR^{d} or oxygen provided that there is no more than one ring oxygen and there are at least two ring heteroatoms and wherein R^{d} is hydrogen or C₁₋₄alkyl;
R² is hydrogen, C₁₋₆alkyl, optionally substituted by hydroxy, cyano or trifluoromethyl, C₂₋₆alkenyl (provided the double bond is not in the 1-position), C₂₋₆alkynyl (provided the triple bond is not in the 1-position), phenylC₁₋₃alkyl or pyridylC₁₋₃alkyl;
R³ is hydrogen, methyl or ethyl;
R⁴ of formula I is optionally substituted: C₁₋₆alkyl, C₃₋₇cycloalkylC₁₋₃alkyl or C₃₋₇cycloalkyl;
or an N-oxide of —NR² where chemically possible;
or an S-oxide of sulphur containing rings where chemically possible;
D of formula II is hydrogen, an optionally substituted 5-7 membered carbocyclic ring containing one double bond, C₁₋₃alkyl substituted by an optionally substituted 5-7 membered carbocyclic ring containing one double bond, or D is of the formula (CH₂)ₙCH(R⁵)C(R⁶)═C(R⁷)R⁸ wherein:
R⁵ is hydrogen, methyl or ethyl;
R⁶ is hydrogen, methyl, bromo, chloro, fluoro or trifluoromethyl;
R⁷ is hydrogen, C₁₋₄alkyl, bromo, chloro, fluoro or trifluoromethyl;
R⁸ is hydrogen, C₁₋₄alkyl, bromo, chloro, fluoro or trifluoromethyl;
and n is 0 or 1;
and N-oxides of —NR² where chemically possible;
and S-oxides of sulfur containing rings where chemically possible;
or a pharmaceutically acceptable salt or in vivo hydrolysable ester or amide thereof.

6. A method of reducing uric acid levels in a warm-blooded animal comprising administering to said animal a therapeutically effective amount of a compound of the formula III; 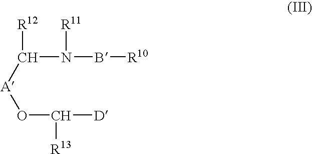
wherein:
A′ is phenyl, naphthyl, pyridyl, pyrazinyl, pyridazinyl, pyrimidyl, thienyl, thiazolyl, oxazolyl, thiadiazolyl having at least two adjacent ring carbon atoms or a bicyclic ring system of the formula: 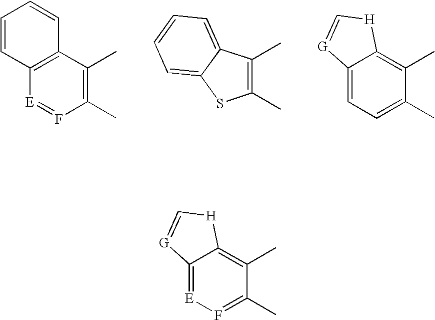
wherein E is nitrogen or CH, F is nitrogen or CH, G is sulphur or oxygen and H is nitrogen or CH,
and wherein A′ is either unsubstituted or substituted by halo, trifluoromethyl, nitro, hydroxy, amino, C₁₋₄alkylamino, diC₁₋₄alkylamino, cyano, C₁₋₆alkoxy, S(O)ₚC₁₋₆alkyl (p is 0, 1 or 2), C₁₋₆alkyl (optionally substituted by hydroxy, amino, halo, nitro or cyano), S(O)ₚCF₃ (p=0, 1 or 2), carbamoyl, C₁₋₄alkylcarbamoyl, di(C₁₋₄alkyl)carbamoyl, C₂₋₆alkenyl, C₂₋₆alkynyl, C₂₋₄alkenylamino, N—C₂₋₄alkenyl-N-C₁₋₄alkylamino, di-C₂₋₄alkenylamino, S(O)ₚC₂₋₆alkenyl, C₂₋₄alkenylcarbamoyl, di-C₂₋₄alkenylcarbamoyl, C₃₋₇cycloalkyl, C₃₋₇cycloalkylC₁₋₃alkyl, C₃₋₇cycloalkylC₂₋₃alkenyl, C₅₋₇cycloalkenyl, C₅₋₇cycloalkenylC₁₋₃alkyl, C₅₋₇cycloalkenylC₂₋₃alkenyl, C₅₋₇cycloalkenylC₂₋₃alkynyl, C₁₋₄alkoxycarbonylamino, C₁₋₄alkanoylamino, C₁₋₄alkanoyl(N—C₁₋₄alkyl)amino, C₁₋₄alkanesulphonamido, benzenesulphonamido, aminosulphonyl, C₁₋₄alkylaminosulphonyl, di(C₁₋₄alkyl)aminosulphonyl, C₁₋₄alkoxycarbonyl, C₁₋₄alkanoyloxy, C₁₋₆alkanoyl, formylC₁₋₄alkyl, trifluoroC₁₋₃alkylsulphonyl, hydroxyiminoC₁₋₆alkyl, C₁₋₄alkoxyiminoC₁₋₆alkyl, C₁₋₆alkylcarbamoylamino, oxazolyl, pyridyl, thiazolyl, pyrimidyl, pyrazinyl or pyridazinyl;
provided that the —CH(R¹²)N(R¹¹)B′—R¹⁰ and —OCH(R¹³)-D′ linking groups are positioned in a 1,2 relationship to one another on ring carbon atoms and the ring atom positioned ortho to the —OCHR¹³— linking group (and therefore in the 3-position relative to the —CHR¹²NR¹¹— linking group) is not substituted;
B′ is phenyl, pyridyl, thiazolyl, oxazolyl, thienyl, thiadiazolyl, isoxazole, pyrazole, furyl, pyrrolyl, imidazolyl, pyrazinyl, pyridazinyl, pyrimidyl, pyridone, pyrimidone, pyrazinone or pyridazinone,
and wherein B′ is either unsubstituted or substituted by amino, C₁₋₄alkylamino, di(C₁₋₄alkyl)amino, halo, trifluoromethyl, nitro, hydroxy, C₁₋₆alkoxy, C₁₋₆alkyl, cyano, —S(O)ₚC₁₋₆alkyl (p is 0, 1 or 2), carbamoyl, C₁₋₄alkylcarbamoyl or di(C₁₋₄alkyl)carbamoyl;
D′ is pyridyl, pyrazinyl, pyrimidinyl, pyridazinyl, pyrrolyl, thienyl, furyl, imidazolyl, pyrazolyl, thiazolyl, isothiazolyl, oxazolyl, isoxazolyl or phenyl,
and wherein D′ is optionally substituted by 1 or 2 substituents selected from halo, trifluoromethyl, nitro, hydroxy, amino, C₁₋₄alkylamino, di(C₁₋₄alkyl)amino, cyano, C₁₋₆alkoxy, —S(O)ₚC₁₋₄alkyl (p is 0, 1 or 2), C₁₋₄alkanoyl, C₁₋₆alkyl, C₃₋₇cycloalkyl, C₃₋₇cycloalkylC₁₋₃alkyl, C₃₋₇cycloalkylC₂₋₃alkenyl, C₅₋₇cycloalkenyl, C₅₋₇cycloalkenylC₁₋₃alkyl, C₅₋₇cycloalkenylC₂₋₃alkenyl, wherein C₃₋₇cycloalkyl, C₅₋₇cycloalkenyl, C₁₋₆alkyl and C₁₋₆alkoxy are optionally substituted by trifluoromethyl, hydroxy, halo, nitro, cyano or amino;
R¹⁰ is positioned on ring B′ in a 1, 3 or 1,4 relationship with the —CH(R¹²)N(R¹¹)— linking group in 6-membered rings and in a 1,3-relationship with the —CH(R¹²)N(R¹¹)— linking group in 5-membered rings and is carboxy, carboxyC₁₋₃alkyl, tetrazolyl, tetrazolylC₁₋₃alkyl, tetronic acid, hydroxamic acid, sulphonic acid, or R¹⁰ is of the formula (IIIA), (IIIB) or (IIIC): 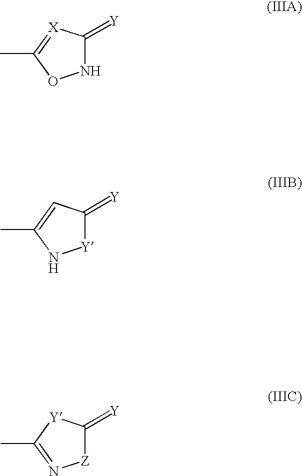
wherein X is CH or nitrogen, Y is oxygen or sulphur, Y¹ is oxygen or NH, and Z is CH₂, NH or oxygen provided that there is no more than one ring oxygen and there are at least two ring heteroatoms;
or R¹⁰ is of the formula —CONR^{e} R^{e1} or —C₁₋₃alkylCONR^{e}R^{e1} wherein R^{e} is hydrogen, C₁₋₆alkyl, C₃₋₇cycloalkyl, C₃₋₇cycloalkylC₁₋₃alkyl, C₅₋₇cycloalkenyl or C₅₋₇cycloalkenylC₁₋₃alkyl and R^{e1} is hydrogen, hydroxy or optionally substituted: C₁₋₁₀alkyl, C₂₋₁₀alkenyl, C₂₋₁₀alkynyl, C₃₋₇cycloalkyl, C₃₋₇cycloalkylC₁₋₆alkyl, C₃₋₇cycloalkylC₂₋₆alkenyl, C₃₋₇cycloalkylC₂₋₆alkynyl, C₅₋₇cycloalkenyl, C₃₋₇cycloalkenylC₁₋₆alkyl, C₅₋₇cycloalkenylC₂₋₆alkenyl, C₅₋₇cycloalkenylC₂₋₆alkynyl, 5- or 6-membered heteroaryl, 5- or 6-membered heteroarylC₁₋₆alkyl, 5- or 6-membered saturated or partially saturated heterocyclyl or 5- or 6-membered saturated or partially saturated heterocyclylC₁₋₆alkyl, and wherein optional substituents on R^{e1} comprise those listed above for ring A′; or wherein R^{e} and R^{e1} together with the amide nitrogen to which they are attached (NR^{e}R^{e1}) form an amino acid residue or ester thereof;
or R¹⁰ is of the formula —CONHSO₂R^{f} or —C₁₋₃alkylCONHSO₂R^{f} wherein R^{f} is optionally substituted: C₁₋₁₀alkyl, C₂₋₁₀alkenyl, C₂₋₁₀alkynyl, C₃₋₇cycloalkyl, C₃₋₇cycloalkylC₁₋₆alkyl, C₃₋₇cycloalkylC₂₋₆alkenyl, C₃₋₇cycloalkylC₂₋₆alkynyl, C₅₋₇cycloalkenyl, C₃₋₇cycloalkenylC₁₋₆alkyl, C₅₋₇cycloalkenylC₂₋₆alkenyl, C₅₋₇cycloalkenylC₂₋₆alkynyl, 5- or 6-membered heteroaryl, 5- or 6-membered heteroylarC₁₋₆alkyl, phenyl, phenylC₁₋₆alkyl, 5- or 6-membered saturated or partially saturated heterocyclyl or 5- or 6-membered saturated or partially saturated heterocyclylC₁₋₆alkyl, and wherein optional substituents on R^{f} comprise those listed above for ring A′;
or R¹⁰ is of the formula —CONR^{e}N(R^{g})R^{h} or —C₁₋₁₃alkylCONR^{e}N(R^{g})R^{h} wherein R^{e} is as hereinabove defined, R^{g} is hydrogen or C₁₋₆alkyl and R^{h} is hydrogen, hydroxy or optionally substituted: C₁₋₁₀alkyl, C₂₋₁₀alkenyl, C₂₋₁₀alkynyl, C₃₋₇cycloalkyl, C₃₋₇cycloalkylC₁₋₆alkyl, C₃₋₇cycloalkylC₂₋₆alkenyl, C₃₋₇cycloalkylC₂₋₆alkynyl, C₅₋₇cycloalkenyl, C₅₋₇cycloalkenylC₁₋₆alkyl, C₅₋₇cycloalkenylC₂₋₆alkenyl, C₅₋₇cycloalkenylC₂₋₆alkynyl, 5- or 6-membered heteroaryl, 5- or 6-membered heteroarylC₁₋₆alkyl, 5- or 6-membered saturated or partially saturated heterocyclyl, 5- or 6-membered saturated or partially saturated heterocyclylC₁₋₆alkyl, and wherein optional substituents on R^{h} comprise those listed above for ring A′; or R^{g} and R^{h}, together with the nitrogen atom to which they are attached, form a 4 to 8-membered saturated or partially saturated heterocyclic ring or form an amino acid residue or ester thereof;
R¹¹ is hydrogen, C₁₋₆alkyl (optionally substituted by hydroxy, cyano, nitro, amino, halo, C₁₋₄alkanoyl, C₁₋₄alkoxy or trifluoromethyl) C₂₋₆alkenyl, C₂₋₆alkynyl, C₃₋₆cycloalkyl, C₃₋₆cycloalkylC₁₋₃alkyl, C₃₋₆cycloalkylC₂₋₃alkenyl, C₅₋₆cycloalkenyl, C₅₋₆cycloalkenylC₁₋₃alkyl, C₅₋₆cycloalkenylC₂₋₃alkenyl, phenylC₁₃alkyl or 5- or 6-membered heteroarylC₁₋₃alkyl;
R¹² is hydrogen or C₁₋₄alkyl;
R¹³ is hydrogen or C₁₋₄alkyl;
or N-oxides of —NR¹¹ where chemically possible;
or S-oxides of sulphur containing rings where chemically possible;
or a pharmaceutically acceptable salt or an in vivo hydrolysable ester or amide thereof; provided that when ring B′ is optionally substituted phenyl and R¹⁰ is an amide of formula —CONR^{e}R^{e1} wherein R^{e} is hydrogen or C₁₋₆alkyl and R^{e1} is hydrogen, then ring B′ does not bear more than one optional substituent.

7. A method of reducing uric acid levels in a warm-blooded animal comprising administering to said animal a therapeutically effective amount of a benzene sulphonamide compound represented by general formula (IV), non-toxic salt thereof or cyclodextrin inclusion complex: 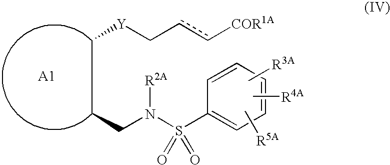
wherein, in formula IV, A1 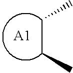
denotes a group represented by the following formulae: 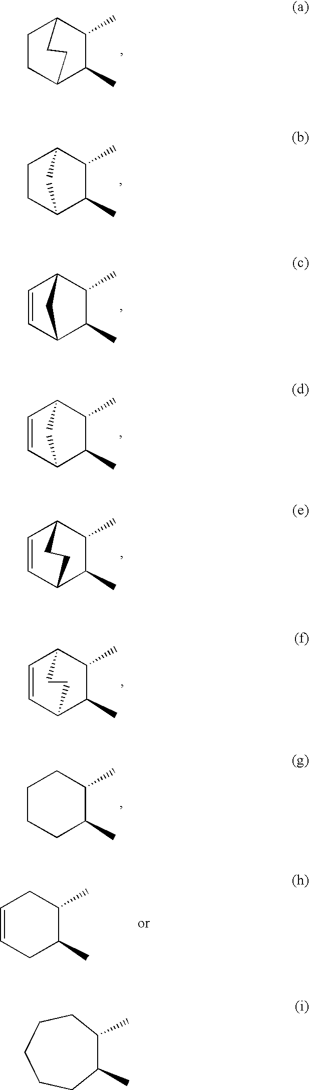
wherein, R^{1A} denotes a hydroxy, alkoxy group of C₁-C₄ or a group represented by general formula NR^{6A}R^{7A};
R^{6A} and R^{7A} each independently represent hydrogen atom or alkyl group of C₁-C₄;
R^{2A} denotes hydrogen atom or alkyl group of C₁-C₄;
R^{3A} and R^{4A} denotes alkyl group of C₁-C₄, halogen atom or trifluoromethyl group;
R^{5A} denotes hydrogen atom, alkyl group of C₁-C₄, halogen atom or trifluoromethyl group;
Y denotes cis-vinylene or trans-vinylene,
and the symbol [symbol] denotes single bond or double bond.

8. A method of reducing uric acid levels in a warm-blooded animal comprising administering to said animal a therapeutically effective amount of a compound represented by general formula (V), ox a carboxamide or a non-toxic salt thereof: 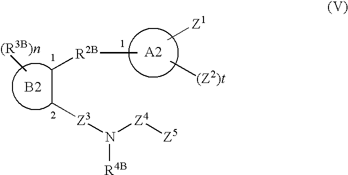
In the formula, ring A² and ring B², each independently denote a carbon-ring of C₅₋₁₅ or 5-7 membered heterocyclic ring having 1 or 2 oxygen, sulfur or nitrogen atoms,
Z¹ comprises a group represented by
(1) —COR^{1B}.
(2) —C₁₋₄ alkylene-COR^{1B}.
(3) —CH═CH—COR^{1B}.
(4) —C≡C—COR^{1B}.
(5) —CO—C₁₋₃ alkylene-COR^{1B};
In the formula, R^{1B} denotes a hydroxy group, C₁₋₄ alkoxy or a group represented by formula NR^{6B}R^{7B} (wherein, R^{6B} and R^{7B} independently denotes hydrogen or C₁₋₄ alkyl, or
(6) —C₁₋₅ alkylene-OH,
Z² denotes hydrogen atom, C₁₋₄ alkyl, C₁₋₄ alkoxy, nitro, halogen, trifluoromethyl, trifluoro methoxy, hydroxy group or a group represented by formula COR^{1B}, wherein, R^{1B} has the same aforesaid meaning),
Z³ represents single bond or C₁₋₄ alkylene,
Z⁴ represents SO₂ or CO,
Z⁵ denotes
(1) C₁₋₈ alkyl, C2-8 alkenyl or C₂₋₈ alkynyl,
(2) phenyl, C₃₋₇ cycloalkyl or 5-7 membered hetero-ring having 1 or 2 oxygen, sulfur or nitrogen atoms,
(3) phenyl or C₃₋₇ cycloalkyl-substituted C₁₋₄ alkyl, C₂₋₄ alkenyl or C₂₋₄ alkynyl;
In aforesaid (2) and (3), phenyl, C₃₋₇ cycloalkyl, 5-7 membered hetero-ring having 1 or 2 oxygen, sulfur or nitrogen atoms, may be substituted with 1-5 R^{5B} groups wherein a plurality of R^{5B} groups independently denote hydrogen atom, C₁₋₆ alkyl, C₁₋₆ alkoxy, C₁₋₆ alkylthio, nitro, halogen, trifluoromethyl, trifluoro methoxy or hydroxy groups;
R^{2B} denotes
CONR^{8B}, NR^{8B}CO,
CONR^{8B}—C₁₋₄ alkylene, C₁₋₄ alkylene-CONR^{8B}, NR^{8B}CO—C₁₋₄ alkylene, C₁₋₄ alkylene-NR^{8B}CO, C₁₋₃ alkylene-CONR^{8B}C₁₋₃ alkylene, C₁₋₃ alkylene-NR^{8B}CO—C₁₋₃ alkylene wherein in each formula, R^{8B} denotes a hydrogen atom or C₁₋₄ alkyl), O, S, NZ⁶, wherein, Z⁶ denotes hydrogen atom or C₁₋₄ alkyl),
Z⁷-C₁₋₄ alkylene,
C₁₋₄ alkylene-Z⁷,
a group represented by C₁₋₃ alkylene-Z⁷-C₁₋₃ alkylene;
wherein, Z⁷ denotes O, S or NZ⁶, wherein, Z⁶ has the same aforesaid meaning;
CO, CO—C₁₋₄ alkylene, C₁₋₄ alkylene-CO, C₁₋₃ alkylene-CO—C₁₋₃ alkylene, C₂₋₄ alkylene, C₂₋₄ alkenylene or C₂₋₄ alkynylene,
R^{3B} denotes hydrogen atom, C₁₋₆ alkyl, C₁₋₆ alkoxy, C₁₋₆ alkylthio, nitro, halogen, trifluoromethyl, trifluoro methoxy, hydroxy group or hydroxymethyl,
R^{4B} denotes
(1) hydrogen atom,
(2) C₁₋₈ alkyl, C₂₋₈ alkenyl or C₂₋₈ alkynyl,
(3) 1 or 2 COOZ⁸, CONZ⁹Z¹⁰, OZ⁸ group, in each group, Z⁸, Z⁹, Z¹⁰ each independently denotes hydrogen atom, C₁₋₄ alkyl and C₁₋₆ alkyl substituted by the group selected from the group comprising C₁₋₄ alkoxy-C₁₋₄ alkoxy,
(4) C₃₋₇ cycloalkyl,
(5) phenyl or C₃₋₇ cycloalkyl-substituted C₁₋₄ alkyl, C₂₋₄ alkenyl or C₂₋₄ alkynyl;
wherein, in the aforesaid (4) and (5), phenyl, C₃₋₇ cycloalkyl may be substituted with 1-5 R^{5B} groups, wherein R^{5B} has the same aforesaid meaning;
n and t respectively, independently represent an integer of 1-4.
Wherein,
(1) R2 and R3 each bond only at 1 and 2 position of ring B2,
(2) when ring A2 represents a benzene ring
and (Z²)ₜ does not represent COR^{1B}, Z¹ is bonded only at 3 or 4 position of said benzene ring.

9. A method of reducing uric acid levels in a warm-blooded animal such as a human being requiring such treatment, which comprises administering to said animal a therapeutically effective amount of a compound selected from the group consisting of:
6-[N-(2-benzyloxy-5-bromobenzyl)-N-ethylamino]pyridazine-3-carboxylic acid,
6-[N-(5-bromo-2-(2-methylprop-2-en-1-yloxy)benzyl)-N-ethylamino]pyridazine-3-carboxylic acid,
N-propanesulphonyl-6-[N-(5-bromo-2-(cyclopropylmethoxy)benzyl)-N-ethylamino]pyridazine-3-carboxamide,
N-(3,5-dimethylisoxazol-4-ylsulphonyl)-6-[N—(S-chloro-2-(2-methylpropoxy)benzyl)-N-ethylamino]pyridazine-3-carboxamide,
6-[N-(5-bromo-2-(cyclopropylmethoxy)benzyl)-N-ethylamino]pyridazine-3-carboxylic acid,
6-[(2S,3 S)-3-(4-chloro-2-methylphenylsulfonylaminomethyl)-bicyclo[2.2.2]octan-2-yl]-5Z-hexenoic acid and
4-[2-[N-isobutyl-N-(2-furylsulfonyl)amino]-5-trifluoromethylphenoxymethyl]cinnamic acid;
or a pharmaceutically acceptable salt or an in vivo hydrolysable ester thereof.

10. A method of reducing uric acid levels in a warm-blooded animal such as a human being requiring such treatment, which comprises administering to said animal a therapeutically effective amount of a compound selected from the group consisting of:
N-propanesulphonyl-6-[N-(5-bromo-2-(cyclopropylmethoxy)benzyl)-N-ethylamino]pyridazine-3-carboxamide, or a pharmaceutically acceptable salt or an in vivo hydrolysable ester thereof.

11. The use of a compound of any one of claims 1, 5, 6, 7, 8 or 9, of Formula I, Formula II, Formula III, Formula IV or Formula V or a pharmaceutically acceptable salt or an in vivo hydrolysable ester thereof, for the manufacture of a medicament for use in the reduction of uric acid in a warm-blooded animal.

12. A compound of any one of claims 1, 5, 6, 7, 8 or 9, of Formula I, Formula II, Formula III, Formula IV or Formula V, or a pharmaceutically acceptable salt or an in vivo hydrolysable ester thereof for use in the reduction of uric acid in a warm-blooded animal.

13. The use of an EP1 antagonist, or a pharmaceutically acceptable salt or an in vivo hydrolysable ester thereof, for the manufacture of a medicament for use in the reduction of uric acid in a warm-blooded animal.

14. An EP1 antagonist, or a pharmaceutically acceptable salt or an in vivo hydrolysable ester thereof for use in the reduction of uric acid in a warm-blooded animal.
